SEQUENCE LISTING (4 sequences)

SEQ ID NO: 1 - DNA, 1473 bp, Homo sapiens
atgcaaccaatcctgcttctgctggccttcctcctgctgcccagggcagatgcaggggagatcatcgggggacatgaggccaagccccactcccgcccctacatggcttatcttatgatctgggatcagaagtctctgaagaggtgcggtggcttcctgatacgagacgacttcgtgctgacagctgctcactgttggggaagctccataaatgtcaccttgggggcccacaatatcaaagaacaggagccgacccagcagtttatccctgtgaaaagacccatcccccatccagcctataatcctaagaacttctccaacgacatcatgctactgcagctggagagaaaggccaagcggaccagagctgtgcagcccctcaggctacctagcaacaaggcccaggtgaagccagggcagacatgcagtgtggccggctgggggcagacggcccccctgggaaaacactcacacacactacaagaggtgaagatgacagtgcaggaagatcgaaagtgcgaatctgacttacgccattattacgacagtaccattgagttgtgcgtgggggacccagagattaaaaagacttcctttaagggggactctggaggccctcttgtgtgtaacaaggtggcccagggcattgtctcctatggacgaaacaatggcatgcctccacgagcctgcaccaaagtctcaagctttgtacactggataaagaaaaccatgaaacgctacgcggccgctggaggcgggggttctggcgggggtggatcagggggtggaggctccggtggaggcgggtcgggcgcgcccgatcctgatgatgttgttgattcttctaaatcttttgtgatggaaaacttttcttcgtaccacgggactaaacctggttatgtagattccattcaaaaaggtatacaaaagccaaaatctggtacacaaggaaattatgacgatgattggaaagggttttatagtaccgacaataaatacgacgctgcgggatactctgtagataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgctgaggggagttctagcgttgaatatattaataactgggaacaggcgaaagcgttaagcgtagaacttgagattaattttgaaacccgtggaaaacgtggccaagatgcgatgtatgagtatatggctcaagcctgtgcaggaaatcgtgtcaggcgatctctttgtgaaggaaccttacttctgtggtgtgacataattggacaaactacctacagagatttaaagctctaa

SEQ ID NO: 2 - DNA, 1623 bp, Homo sapiens
atgcaaccaatcctgcttctgctggccttcctcctgctgcccagggcagatgcaggggagatcatcgggggacatgaggccaagccccactcccgcccctacatggcttatcttatgatctgggatcagaagtctctgaagaggtgcggtggcttcctgatacgagacgacttcgtgctgacagctgctcactgttggggaagctccataaatgtcaccttgggggcccacaatatcaaagaacaggagccgacccagcagtttatccctgtgaaaagacccatcccccatccagcctataatcctaagaacttctccaacgacatcatgctactgcagctggagagaaaggccaagcggaccagagctgtgcagcccctcaggctacctagcaacaaggcccaggtgaagccagggcagacatgcagtgtggccggctgggggcagacggcccccctgggaaaacactcacacacactacaagaggtgaagatgacagtgcaggaagatcgaaagtgcgaatctgacttacgccattattacgacagtaccattgagttgtgcgtgggggacccagagattaaaaagacttcctttaagggggactctggaggccctcttgtgtgtaacaaggtggcccagggcattgtctcctatggacgaaacaatggcatgcctccacgagcctgcaccaaagtctcaagctttgtacactggataaagaaaaccatgaaacgctacgcggccgctggaggcgggggttctggcgggggtggatcagggggtggaggctccggtggaggcgggtcgggcgcgcccaaacgccacacgcgtacgcgggtcacatcaatcacattagatctagtaaatccgaccgcgggtcaatactcatcttttgtggataaaatccgaaacaacgtaaaggatccaaacctgaaatacggtggtaccgacatagccgtgataggcccaccttctaaagaaaaattccttagaattaatttccaaagttcccgaggaacggtctcacttggcctaaaacgcgataacttgtatgtggtcgcgtatcttgcaatggataacacgaatgttaatcgggcatattacttcaaatcagaaattacttccgccgagttaaccgcccttttcccagaggccacaactgcaaatcagaaagctttagaatacacagaagattatcagtcgatcgaaaagaatgcccagataacacagggagataaaagtagaaaagaactcgggttggggatcgacttacttttgacgttcatggaagcagtgaacaagaaggcacgtgtggttaaaaacgaagctaggtttctgcttatcgctattcaaatgacagctgaggtagcacgatttaggtacattcaaaacttggtaactaagaacttccccaacaagttcgactcggataacaaggtgattcaatttgaagtcagctggcgtaagatttctacggcaatatacggggatgccaaaaacggcgtgtttaataaagattatgatttcgggtttggaaaagtgaggcaggtgaaggacttgcaaatgggactccttatgtatttgggcaaaccaaaggcctccgaaaaggacgagttatag

SEQ ID NO: 3 - DNA, 1848 bp, Homo sapiens
atgcaaccaatcctgcttctgctggccttcctcctgctgcccagggcagatgcaggggagatcatcgggggacatgaggccaagccccactcccgcccctacatggcttatcttatgatctgggatcagaagtctctgaagaggtgcggtggcttcctgatacgagacgacttcgtgctgacagctgctcactgttggggaagctccataaatgtcaccttgggggcccacaatatcaaagaacaggagccgacccagcagtttatccctgtgaaaagacccatcccccatccagcctataatcctaagaacttctccaacgacatcatgctactgcagctggagagaaaggccaagcggaccagagctgtgcagcccctcaggctacctagcaacaaggcccaggtgaagccagggcagacatgcagtgtggccggctgggggcagacggcccccctgggaaaacactcacacacactacaagaggtgaagatgacagtgcaggaagatcgaaagtgcgaatctgacttacgccattattacgacagtaccattgagttgtgcgtgggggacccagagattaaaaagacttcctttaagggggactctggaggccctcttgtgtgtaacaaggtggcccagggcattgtctcctatggacgaaacaatggcatgcctccacgagcctgcaccaaagtctcaagctttgtacactggataaagaaaaccatgaaacgctacgcggccgctggaggcgggggttctggcgggggtggatcagggggtggaggctccggtggaggcgggtcgggcgcgccctccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattaa

SEQ ID NO: 4 - DNA, 1944 bp, Homo sapiens
atgcaaccaatcctgcttctgctggccttcctcctgctgcccagggcagatgcaggggagatcatcgggggacatgaggccaagccccactcccgcccctacatggcttatcttatgatctgggatcagaagtctctgaagaggtgcggtggcttcctgatacgagacgacttcgtgctgacagctgctcactgttggggaagctccataaatgtcaccttgggggcccacaatatcaaagaacaggagccgacccagcagtttatccctgtgaaaagacccatcccccatccagcctataatcctaagaacttctccaacgacatcatgctactgcagctggagagaaaggccaagcggaccagagctgtgcagcccctcaggctacctagcaacaaggcccaggtgaagccagggcagacatgcagtgtggccggctgggggcagacggcccccctgggaaaacactcacacacactacaagaggtgaagatgacagtgcaggaagatcgaaagtgcgaatctgacttacgccattattacgacagtaccattgagttgtgcgtgggggacccagagattaaaaagacttcctttaagggggactctggaggccctcttgtgtgtaacaaggtggcccagggcattgtctcctatggacgaaacaatggcatgcctccacgagcctgcaccaaagtctcaagctttgtacactggataaagaaaaccatgaaacgctacgcggccgctggaggcgggggttctggcgggggtggatcagggggtggaggctccggtggaggcgggtcgggcgcgcccgcttcgtacccctgccatcaacacgcgtctgcgttcgaccaggctgcgcgttctcgcggccatagcaaccgacgtacggcgttgcgccctcgccggcagcaagaagccacggaagtccgcctggagcagaaaatgcccacgctactgcgggtttatatagacggtcctcacgggatggggaaaaccaccaccacgcaactgctggtggccctgggttcgcgcgacgatatcgtctacgtacccgagccgatgacttactggcaggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcgataccttatgggcagcatgaccccccaggccgtgctggcgttcgtggccctcatcccgccgaccttgcccggcacaaacatcgtgttgggggcccttccggaggacagacacatcgaccgcctggccaaacgccagcgccccggcgagcggcttgacctggctatgctggccgcgattcgccgcgtttacgggctgcttgccaatacggtgcggtatctgcagggcggcgggtcgtggcgggaggattggggacagctttcggggacggccgtgccgccccagggtgccgagccccagagcaacgcgggcccacgaccccatatcggggacacgttatttaccctgtttcgggcccccgagttgctggcccccaacggcgacctgtacaacgtgtttgcctgggccttggacgtcttggccaaacgcctccgtcccatgcacgtctttatcctggattacgaccaatcgcccgccggctgccgggacgccctgctgcaacttacctccgggatggtccagacccacgtcaccacccccggctccataccgacgatctgcgacctggcgcgcacgtttgcccgggagatgggggaggctaac

(19) United States
(12) Patent Application Publication
(10) Pub. No.: US 2017/0182096 A1
(43) Pub. Date: Jun. 29, 2017

(54) LYMPHOCYTE MEDIATED DELIVERY OF PROTEINS

(71) Applicant: BRITISH COLUMBIA CANCER AGENCY BRANCH, Vancouver (CA)

(72) Inventors: Robert Holt, Vancouver (CA); Daniel Woodsworth, Vancouver (CA)

(21) Appl. No.: 15/301,343

(22) PCT Filed: Apr. 15, 2015
(86) PCT No.: PCT/CA2015/050312

§ 371 Date: Sep. 30, 2016

Related U.S. Application Data
(60) Provisional application No. 61/980,341, filed on Apr. 16, 2014.

(51) Int. Cl. A61K 35/17; A61K 38/45; C12N 5/0783; C12N 9/64; C12N 9/24; C12N 9/10; A61K 38/47; A61K 38/48

(52) U.S. Cl. CPC A61K 35/17; A61K 38/47; A61K 38/45; A61K 38/482; C12Y 304/21079; C12N 9/6467; C12N 9/2497; C12N 9/1077; C12Y 302/02022; C12Y 204/02036; C12N 5/0646; C12N 2740/15043; C12N 2510/00

(57) ABSTRACT

The invention is directed to methods and compositions for cell-based targeted delivery of predetermined compounds to a population of target cells. In some embodiments, methods of the invention include providing cytotoxic lymphocytes genetically modified to produce and sequester in lytic granules fusion proteins comprising a granzyme, or other effector agent, and a predetermined protein, so that upon specific contact of the cytotoxic lymphocytes with the target cells, the granzyme-perforin pathway of the cytotoxic lymphocytes is activated, leading to the delivery of the fusion protein to the cytosols of the target cells.

### FIELD OF THE INVENTION

This invention relates generally to cell-based delivery systems, and more particularly, to the targeted delivery of compounds to selected cell populations using the granzyme-perforin pathway and modifications thereof.

### BACKGROUND

Engineered cell-based therapeutics provide promising new approaches to treating complex diseases because of a cell's ability to sense and integrate a wide range of signals, actively move to specific tissue compartments, and actuate context-dependent responses, e.g. Fischbach et al, Science Transl. Med., 5: 179ps7 (2013). Such cell-based approaches provide novel therapeutic devices that address current obstacles faced by small molecules and biologics, such as poor target specificity, undesirable tissue compartment localization, a lack of personalization, and limited potential for the effects of the drug to be modified once administered to a patient, either over space and time, or in response to a changing clinical picture. These issues can result in limited pharmaceutical utility due to significant morbidity and mortality, sub-optimal dosing, limited therapeutic index, or poor patient compliance. Cytotoxic lymphocytes (CLs), such as cytotoxic T-lymphocytes (CTLs) and Natural Killer cells (NKs), are an excellent platform for engineering cell-based therapeutic systems for several reasons: (i) cytotoxic lymphocytes possess a unique delivery-cell-to-target-cell molecular transfer system in the granzyme-perforin pathway; (ii) T-cell receptors (TCRs), or the related chimeric antigen receptors (CARs), endow cytotoxic lymphocytes with an exquisite level of specificity in targeting a cell population presenting Major Histocompatibility Complex (MHC) bound cognate antigen or, in the case of CARs, an arbitrary surface antigen; (iii) activated cytotoxic lymphocytes differentially express cytokine and tissue specific receptors, enabling selective lymphocyte homing throughout the body to target tissue; and (iv) laboratory and clinical protocols for lymphocyte modification and therapeutic administration have been developed in the field of adoptive cell therapy, e.g., Restifo et al, Nature Reviews Immunology, 12: 269-281 (2012).

Cell-based therapeutic approaches have been pursued as cancer treatments. For example, it is well known that cancer cells frequently elude the immune response. This is partially due to tumor cell evasion of cytotoxic lymphocyte recognition, primarily via down-regulation of antigen processing and surface MHC expression (for CTL targeting) and up-regulation of inhibitory receptors (for NK attack). The advent of chimeric antigen receptor therapy has begun to address this aspect of the above problem, e.g. Brentjens et al, Science Translational Medicine, 5: 177ra38 (2013); Porter et al, New England J. Medicine, 365: 725-733 (2011). However, independent of evading recognition by lymphocytes, tumour cells have been shown to be highly resistant to lymphocyte cytotoxic effector mechanisms. Disruption of apoptosis pathways mediated by death receptors (such as the Fas system, an ancillary NK apoptotic effector mechanism) has been found across a range of cancers, and has been implicated in carcinogenesis as well as apoptosis resistance, e.g. Debatin, Cancer Immunology, Immunotherapy: CII, 53: 153-159 (2004). Down-regulation of the executioner caspases 3 and 7 is widely observed and correlates with poor survival, e.g. Devarajan et al, Oncogene, 21: 8843-8851 (2002). Inhibitor of apoptosis proteins (IAPs) are consistently overexpressed in tumors, have been shown to initiate hematological malignancies in vivo, are responsible for metastatic potential, have been found to cause resistance to adoptively transferred lymphocytes, and are being actively pursued as small molecule targets, with these efforts having progressed to clinical trials, e.g. Fulda et al, Nature Reviews Drug Discovery, 11: 109-124 (2012). Direct inhibition of granzyme by overexpressed serpins is well characterized. Most importantly, overexpression of XIAP, survivin, and serpinb9 confer apoptosis resistance to tumor cells, disrupt key nodes in apoptotic pathways and are directly and specifically responsible for the resistance of these cells to lymphocyte mediated cytotoxicity, despite effective targeting, both in vitro and in vivo, e.g. Bots et al, Immunology and Cell Biology, 84: 79-86 (2006). Thus, apoptosis resistance and specifically resistance to lymphocyte-induced apoptosis is one of several unsolved therapeutic challenges that may be amenable to solution by cell-based approaches.

In view of the above, the availability of new methods and compositions for cell-based targeted delivery of predetermined compounds, such as proteins, to specified populations of cells would be advantageous for several fields, including but not limited to, cell-based therapeutics, in vivo diagnostics, monitoring, imaging, and the like. In particular, the availability of new methods and compositions for specific delivery of therapeutic proteins to tumor cells with apoptosis resistance would be a major advance in cancer therapy.

### SUMMARY OF THE INVENTION

The present invention is directed to methods and compositions for delivering compounds, such as predetermined proteins, for example, therapeutic proteins, to specific target cells. Aspects of the present invention are exemplified in a number of implementations and applications, some of which are summarized below and throughout the specification.

In one aspect, the invention includes a method of delivering a compound to target cells comprising the steps of (a) providing cytotoxic lymphocytes specific for the target cells and having a granzyme-perforin pathway; (b) genetically modifying the cytotoxic lymphocytes to express a fusion protein comprising an effector agent, such as a granzyme, and a predetermined protein to form a population of delivery lymphocytes, such that the fusion protein is sequestered in lytic granules of the delivery lymphocyte; (c) contacting the target cells with the delivery lymphocytes so that granzyme-perforin pathways thereof are activated, thereby delivering the fusion protein to cytosols of the target cells. In some embodiments of this aspect, the method includes providing cytotoxic lymphocytes specific for target cells of an individual and includes administering the cytotoxic lymphocytes to the individual so that they contact the target cells of the individual.

In another aspect, the invention is directed to a composition for delivering a predetermined protein to target cells that comprises cytotoxic lymphocytes specific for the target cells, the cytotoxic lymphocytes being genetically modified to express a fusion protein comprising an effector agent and a predetermined protein, wherein the fusion protein is sequestered in lytic granules of the cytotoxic lymphocyte and deliverable to cytosols of the target cells by a granzyme-perforin pathway of the cytotoxic lymphocytes whenever the cytotoxic lymphocytes contact the target cells.

In one embodiment of the present invention, granzyme protein is modified to act as a carrier to deliver proteins or peptides of interest to target cells in an antigen specific manner. In a further aspect of the present invention a modified granzyme gene is introduced into adaptive (Cytotoxic T Lymphocytes (CTL)) or innate (Natural Killer (NK)) cytotoxic lymphocytes and said lymphocytes are then introduced into an organism in order to deliver compounds, such as proteins or peptides of interest, to target cells in an antigen specific manner.

In one embodiment of the present invention, an effector agent, such as a lytic granule protein, including, but not limited to, granzyme A, granzyme B, granzyme H, granzyme K or granzyme M, or granulysin, serglycin, or the like, is modified to deliver a toxin for the purpose of killing or ablating a selected target cell population. In another embodiment, a lytic granule protein, such as a granzyme (for example, a granzyme A, granzyme B, granzyme H, granzyme K or granzyme M) is modified to deliver a toxin for the purpose of killing or ablating a selected target cell population. In an alternative embodiment, such effector agent is modified to deliver a functional protein that is deficient or absent from the target cell population. In a further alternative embodiment, such effector agent is modified to deliver a protein which provides a new or additional function, or to modulate an existing function, to a target cell population. In some embodiments, cytotoxic lymphocytes are engineered to deliver such modified effector agents to the selected target cell populations. In some embodiments, such cytotoxic lymphocytes and modified effector agents are from the same mammalian species, for example, human cytotoxic lymphocytes deliver modified human effector agents, mouse cytotoxic lymphocytes deliver modified mouse effector agents, and the like. In some embodiments, a species of cytotoxic lymphocytes may be different than that of a modified effector agent. For example, for research and preclinical testing cytotoxic lymphocytes of experimental mammals may deliver modified human effector agents to target cells of the experimental mammal.

In one aspect of the present invention, the modified granzyme protein is introduced into lymphocytes for the purpose of targeting of cell populations causing disease. In one embodiment, the modified granzyme delivers a toxin in order to kill infected, disregulated, malignant or otherwise harmful cells. In alternate embodiments, the modified granzyme provides a protein or peptide that is absent because of an inherited or acquired genetic variant for metabolic deficiencies and other mendelian diseases. In a still further alternate embodiment, the modified granzyme provides a protein or peptide which is protective or therapeutic for cells in an inflammatory degenerative state, such as in neurodegenerative or auto-immune diseases. In a still further alternative embodiment, the modified granzyme provides a protein or peptide that interacts with endogenous cell signaling networks, for the purpose of inducing a change in cell type, differentiation or function.

In one aspect, the invention addresses an unsolved challenge in the field of cancer therapy by providing methods for killing apoptosis resistant tumor cells. The invention provides a new class of cell-based therapeutics to overcome apoptosis resistant tumor cells by safe, specific delivery of alternative highly toxic molecules to such target cells. In some embodiments, methods of the invention may be used in a stand-alone fashion to target apoptosis-resistant cells that are insensitive to standard treatment, yet remain antigenic. In other embodiments, methods of the invention may be combined with CAR technology, or other antigen- or cell-specific targeting technologies, to eliminate tumor cells that are both treatment resistant and immune evasive.

These above-characterized aspects, as well as other aspects, of the present invention are exemplified in a number of illustrated implementations and applications, some of which are shown in the figures and characterized in the claims section that follows. However, the above summary is not intended to describe each illustrated embodiment or every implementation of the present invention.

### BRIEF DESCRIPTIONS OF THE DRAWINGS

FIG. 1 illustrates diagrammatically aspects of the present invention.

FIGS. 2A-2B illustrate a vector for producing fusion proteins of the invention.

FIG. 2C show data of production of granzyme B-reporter fusion protein using the vector of FIGS. 2A-2B.

FIG. 3 diagrammatically shows elements of a lentivirus vector that may be used in some embodiments of the invention.

FIG. 4 shows FACS data of co-culture experiments to demonstrate transfer of GzB-tdTomato fusion proteins from NK cells to target K562 cells.

FIG. 5 shows data demonstrating resistance to killing of cell systems used for modeling apoptosis resistance. K562 cells stably overexpressing XIAP were co-cultured with CFSE labeled NK-92MI cells at a 4:1 effector to target ratio for 4 hours. These co-cultures were then stained with propidium iodide and analyzed via flow cytometry.

FIGS. 6A and 6B shows data demonstrating mutant EF2 (mEF2) overexpressed in HEK293T cells rescues protein expression from Diphtheria Toxin blockade.

FIGS. 7A and 7B show data demonstrating granzyme fusion proteins with toxin payloads of thymidine kinase (TK) and diptheria toxin (DTA) effectively kill apoptosis resistant cells.

### DETAILED DESCRIPTION OF THE INVENTION

The practice of the present invention may employ, unless otherwise indicated, conventional techniques and descriptions of organic chemistry, molecular biology (including recombinant techniques), cell biology, and biochemistry, which are within the skill of the art. Such conventional techniques include, but are not limited to, preparation of synthetic polynucleotides, monoclonal antibodies, antibody display systems, cell and tissue culture techniques, nucleic acid sequencing and analysis, and the like. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV); PCR Primer: A Laboratory Manual; Retroviruses; and Molecular Cloning: A Laboratory Manual (all from Cold Spring Harbor Laboratory Press); Renault and Duchateau, Editors, Site-directed Insertion of Transgenes (Springer, Heidelberg, 2013); Lutz and Bornscheuer, Editors, Protein Engineering Handbook (Wiley-VCH, 2009); Hermanson, Bioconjugate Techniques, Second Edition (Academic Press, 2008); and the like.

### The Granzyme-Perforin Pathway

In one aspect, the invention provides novel cell-based targeted delivery systems based on the granzyme-perforin pathway. In particular embodiments, the invention provides cells having engineered granzyme-perforin pathways for delivering to target cells predetermined compounds, especially proteins, which may be designed to affect the target cells in a variety of ways; for example, such a predetermined compound may selected to be toxic to the target cells or, in other embodiments, to be beneficial to the target cells. Such cells may be engineered from any cell type that possesses, or can be engineered to possess, a granzyme-perforin pathway and a molecular recognition system for specifically binding to selected target cells. Although reference is made to a “granzyme-perforin pathway,” and equivalently to the “granzyme-perforin system” this is not meant to limit fusion proteins made in accordance with the invention to fusion of a granzyme and a predetermined or selected protein. As discussed more fully below, a granzyme-perforin pathway may deliver fusion proteins comprising any protein effector agent and a predetermined protein. For example, a granzyme-perforin pathway may deliver a fusion protein comprising all or a portion of a granzyme, a granulysin, a serglycin, or other protein that is a component of a lytic granule.

In some embodiments, such cells are engineered from cytotoxic lymphocytes. Cytotoxic lymphocytes are key elements of the immune response that are mainly responsible for the recognition and clearance of cells infected by intracellular pathogens, as well as tumour immunosurveillance. For one type of cytotoxic lymphocytes, Cytotoxic T Lymphocytes (CTL), identification of target cells is a complex process that hinges upon T cell receptor engagement of a cognate peptide presented by cell surface MHC. This interaction activates the key cytotoxic effector mechanisms of CTLs including the granzyme-perforin pathway. In humans there are at least four granzymes (granzyme A, granzyme B, granzyme K and granzyme M) of which granzyme B (GzB) is the best characterized. GzB is a serine protease with a classical trypsin-like catalytic triad that initiates apoptosis in targeted cells. Synthesized primarily in cytotoxic lymphocytes as a 247 amino acid precursor protein, GzB is directed to the endoplasmic reticulum by a signal peptide, which is subsequently cleaved, yielding the zymogen form of GzB, which is still inactive due to an N-terminal dipeptide. This proenzyme is sorted through the Golgi network in a pathway that involves the addition of mannose-6-phosphate, as well as the chaperone molecule serglycin, both of which promote localization of GzB to lytic granules (LGs), a type of specialized secretory lysososome. Here the dipeptide is cleaved by cathepsin C, and the active form of GzB is safely sequestered in the acidic LG and stored there awaiting CTL activation. The other major component of this pathway is perforin, a long, thin protein that forms pores in targeted cells, and is stored in LGs along with granzyme-serglycin aggregates. Upon TCR engagement in an activated CTL, a tight enclosed region between the CTL and target cell forms, which is known as the immunological synapse (IS). Perforin and granzyme are exocytosed from the LGs into the IS, and diffuse across to the target cell membrane, into which perforin inserts, and then aggregates to form multimeric, transmembrane pores. The pores seem to be only briefly patent before membrane integrity is restored, with their main function being a conduit for passive diffusion of effector agents (such as granzyme B and the other granzymes and granulysin which are co-packaged with GzB in LGs) into the target cell. Once in the cytosol, it is the effector agents (the lytic granule proteins including GzB) that initiate apoptosis. GzB cleaves BH3 interacting-domain death agonist (BID) and caspases 3,7 and 8, which in turn activate the mitochondrial and caspase apoptosis pathways respectively. In summary, the synergistic activities of granzyme and perforin represent a unique pathway for transferring molecules from CTLs to target cell exclusively, as the immunological synapse confines granzyme and perforin between the two cells, and moreover, significant numbers of perforin molecules are required to form the pores required for granzyme's entry into the target cell. The granzyme-perforin pathway functions similarly in other types of cytotoxic lymophocytes , such as Natural Killer Cells.

The engineering, or genetic modifications, of cytotoxic lymphocytes may include, but are not limited to, (i) addition of genes for targeting capability, e.g. genes that encode CARs, or like targeting molecules, and (ii) addition of genes that encode one or more fusion proteins each comprising an effector agent or a portion thereof and a predetermined protein (iii) addition of genes that encode proteins for protecting the delivery cell from effects of a predetermined protein, for example when such protein is a toxin, and/or (iv) addition of reporter genes to facilitate tracking and manipulation of the engineered cells. In some embodiments, engineered cells of the invention, i.e. delivery cells, comprise cytotoxic lymphocytes, cytotoxic T cells, NK cells, mast cells, or the like. In other embodiments, engineered cells of the invention comprise cells that are not natural lymphocytes, but a cell type modified to express the some or all of the components of the granzyme-perforin system. In other embodiments, engineered cells of the invention comprise cytotoxic lymphocytes. These different cell types are sometimes referred to herein collectively as “delivery lymphocytes”.

As is also discussed below, a fusion protein in accordance with the invention does not need to include an entire protein effector agent. In some embodiments, a fusion protein may include only a sequence motif from, or a portion of, an effector agent which is necessary to provide sequestration in a lytic granule and transport to the cytosol of a target cell. In other embodiments, an effector agent or portion thereof in a fusion protein may be only related to the natural effector agent by a degree of amino acid homology. For example, such effector agent may have 90 percent, or 80 percent, or 70 percent, or 50 percent, or 40 percent, or 30 percent amino acid homology, or sequence identity, with the effector agent native to a delivery lymphocyte. In some embodiments, only a C-terminal portion of a protein effector agent may comprise a fusion protein; in some embodiments, such C-terminal portion may comprise less than 100 percent, 90 percent, 50 percent, 20 percent, 10 percent or 5 percent of the amino acids of an effector agent. In other embodiments, only an N-terminal portion of a protein effector agent may comprise a fusion protein; in some embodiments, such N-terminal portion may comprise less than 100 percent, 90 percent, 50 percent, 20 percent, 10 percent or 5 percent of the amino acids of an effector agent. In still other embodiments, an internal segment of a protein effector agent may comprise a fusion protein; in some embodiments, such internal segment may comprise less than 100 percent, 90 percent, 50 percent, 20 percent, 10 percent or 5 percent of the amino acids of an effector agent. In further embodiments, such portion or segment is selected so that a fusion protein is sequestered in a lytic granule. In still further embodiments, such portion or segment is selected so that a fusion protein is sequestered in a lytic granule with at least fifty percent of the efficiency of the corresponding natural effector agent.

In some embodiments, a granzyme-perforin pathway comprises (i) lytic granules containing one or more effector agents including perforin, and (ii) an activatable intracellular transport system for moving lytic granules to the delivery cell surface membrane for exocytosis and release of the one or more effector agents, such that released perforins form pores in the surface membrane a recognized target cell. Under these conditions, released non-perforin effector agents, including fusion proteins, are transported by diffusion into the cytosol of the target cells. The activatable intracellular transport system is activated directly or indirectly by specific recognition of a target cell by the delivery cell. Recognition may be accomplished by specific binding of targeting molecules on the surface of the delivery cells to one or more antigens on the surface of the target cells. For example, recognition may be accomplished by specific binding of a TCR on a delivery cell, such as a CTL, to an MHC-epitope complex on the surface of a target cell; or recognition may be accomplished by specific binding of a CAR on a delivery cell to a predetermined antigen of the surface of a target cell. Other molecular or metabolic targeting structures known to those with skill in the art may also be employed with granzyme-perforin pathways using conventional cellular engineering methods.

In one aspect, the invention provides a novel approach to therapeutic molecule delivery by engineering the granzyme-perforin pathway of cytotoxic lymphocytes. In some embodiments, such engineering comprises expressing toxin-effector agent fusion proteins which are sequestered in lytic granules followed by delivery to target cells. When combined with antigen receptor-mediated targeting, methods of the invention provide a new class of cell-based therapeutics with improved efficacy and lower iatrogenic toxicity. The invention is complementary with other cell therapies, including Haematopoietic Stem Cell Transplant (HSCT) and adoptive transfer of tumor reactive lymphocytes including, most recently, Chimeric Antigen Receptor (CAR) engineered lymphocytes. In some embodiments, methods of the invention include antigen receptor mediated targeting and a safety suicide system. In some embodiments, method of the invention provide a capability of cytoplasmic delivery of predetermined protein payloads, for example, toxin-effector agent fusion proteins, in a highly specific manner to a target cell population.

Upon activation of a cytotoxic lymphocyte, effector agents, such as perforin and granzyme are released from the cytoplasmic lytic granules (LGs) in which they are stored, into the immunological synapse that forms between a cytotoxic lymphocyte and its target cell. Perforin inserts into the target cell membrane and aggregates to form multimeric, transmembrane pores. Perforin alone is not cytotoxic at physiological concentrations, rather it functions to permit passive but highly localized diffusion of granzyme into the target cell. Once in the target cytosol granzyme, primarily granzyme B (GzB) in humans, initiates apoptosis by cleavage of BID and caspases 3,7 & 8. In part, the invention provides a method of using this pathway for the purpose of delivering a protein, such as a therapeutic protein, by using GzB, or another lytic granule protein, as a molecular chaperone to carry a predetermined protein into a target cell, such as a tumor cell. In one embodiment, this is achieved by fusing the predetermined protein to the intact GzB protein such that upon transfection of a lymphocyte with a vector encoding the fusion protein, it will be expressed and packaged into lytic granules and released upon specific binding of such lymphocyte to target cells (for example, via T cell and MHC receptors binding, CAR-target binding, or the like).

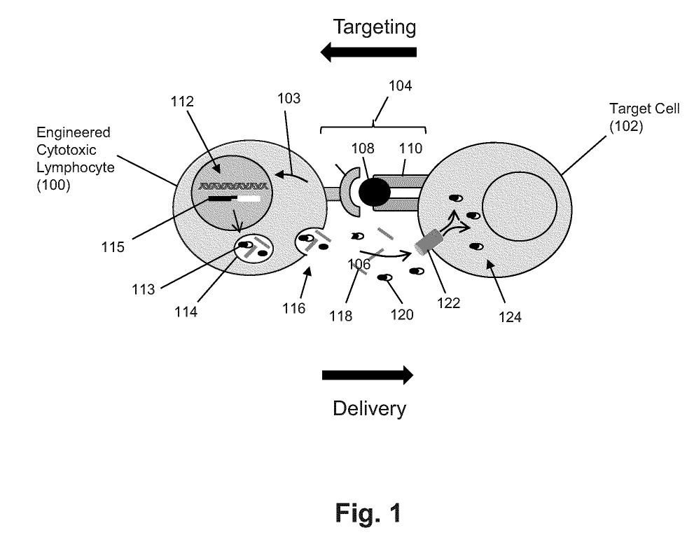

Some of the above aspects of the invention are illustrated diagrammatically in FIG. 1. Target cell (102), for example, a tumor cell, expresses epitope (108) in the context of MHC molecule (110) which is specifically recognized by receptor (106). This is a targeting feature (104) of one embodiment of the invention. In other embodiments, targeting may be accomplished with a chimeric antigen receptor (CAR) expressed by cytotoxic lymphocyte (100), or with other molecular targeting structures. Engineered cytotoxic lymphocyte (100) produces lytic granules (114) in which fusion protein (113) and other effector agents are sequestered. After such targeting and recognition, engineered cytotoxic lymphocyte (100) is activated (103) to produce an effector response (112) that includes transport of lytic granules (114) to the cell membrane where its contents are released by exocytosis (116). (That is, its granzyme-perforin pathway is activated). In some embodiments, cytotoxic lymphocyte (100) is genetically modified to express such fusion proteins by stably integrated genetic element (115), which may be produced, for example, by an integrated viral vector. Such excytotic release includes the release of perforins (118) which assemble into pores (122) in the cell membrane of target cells (102). Fusion proteins (124) along with other effector agents then pass through pore (122) and are delivered into the cytosol of target cells (102).

In some embodiments, this lymphocyte-based delivery system may be used to deliver therapeutic proteins, which may deliver a therapeutic effect directly as a fusion protein, or which may undergo further processing, for example, by auto-cleavage and release from a fusion protein prior to delivering a therapeutic effect. The engineered lymphocytes of such a delivery system may be produced and administered using techniques of Adoptive Cell Therapy (ACT), e.g. Restifo et al, Nature Reviews Immunology, 12: 269-281 (2011), so that lymphocyte mediated delivery of therapeutic proteins provides a new modality for cancer treatment. Refractory cancer often results from loss of response to apoptotic signals, primarily via down regulation of executioner caspases 3 and 7 and/or up-regulation of inhibitor of apoptosis proteins (IAPs). Such forms of resistance have been observed in many malignancies and correlate significantly with poor survival and other negative outcomes, which underscores the need for new therapies. The ability of tumors to evade immune responses remains a significant challenge for cancer therapy. CTLs and NK cells exert their natural cytotoxic effects primarily by delivering effector agents, such as granzymes, via perforin pores to target cells, where, for example, granzymes subsequently cleave target cell caspases to induce target cell apoptosis. However, as mentioned above, tumor cells are often apoptosis resistant, or acquire such resistance. In fact, apoptosis evasion is a hallmark of cancer and chemotherapy resistance. In accordance with some embodiments of the invention, cytotoxic lymphocytes are modified by adding a gene that encodes a fusion protein, such as a modified version of granzyme that has attached to it a therapeutic protein, for example, a highly potent bacterial toxin for enhanced killing of tumor cells by a mechanism that is not dependent on apoptotic mechanisms. In some embodiments, for cancer treatment using this method, cytotoxic lymphocytes are modified with the gene for the highly potent toxin-effector agent combination, and re-administered to the patient. That is, in some embodiments, methods of the invention for treating a cancer of a patient comprise steps of (a) genetically modifying cytotoxic lymphocytes so that they have a capability of expressing at least one toxin-effector agent in lytic granules, (b) expanding the genetically modified cells, and (c) administering the genetically modified cells to the patient. In some such embodiments, the method may include a further step of obtaining cytotoxic lymphocytes from white blood cells of the patient or of MHC-matched donors. In other such embodiments, cytotoxic lymphocytes may be derived from cell lines (e.g. NK-92 cell line, or the like) or engineered cells. In some embodiments, such cytotoxic lymphocytes may be further genetically modified to express a chimeric antigen receptor to direct such lymphocytes to specific target cells, as well as further modifications to enhance persistence, trafficking, targeting, efficacy or safety. In other embodiments, a species-matched cell line may be provided in place of autologous or donor-HLA-matched cytotoxic lymphocytes, wherein such cell line is genetically modified to be HLA-matched as well as to produce a toxin-effector agent.

All current cell therapies for cancer that are being developed and tested rely on the natural cytotoxicity of lymphocytes. That is, the natural degranulation of the lymphocyte against its target tumor cell and granzyme mediated induction of target cell apoptosis. Similarly, most current targeted monoclonal antibody therapeutics for cancer rely, ultimately, on endogenous cytotoxic lymphocytes (either via antibody dependent cellular cytotoxicity, ADCC, or by disinhibiting lymphocytes via checkpoint receptor blockade) for their tumoricidal effects. In accordance with some embodiments of the present invention, by exploiting effector agents of cytotoxic T cells or NK cells as delivery vehicles for an alternative toxic molecule, apoptosis resistance can be circumvented. Antibody-toxin conjugates have direct anti-tumor potential and are a mature technology, however, these interventions are often hampered by limited distribution and target cell internalization, as well as substantial immunogenicity leading to poor bioavailability. In some embodiments with therapeutic applications, (a) a biological cell is used as a delivery vehicle, which has substantial advantages, including (i) protecting the delivered molecule from immunological clearance as well as constitutive breakdown; (ii) enabling antigen mediated selection of targeted tissue; (iii) achieving widespread tissue distribution, (iv) a granzyme-perforin system inherent in these lymphocytes is used to enable extremely specific cell-to-cell delivery of an arbitrary predetermined protein, while all but eliminating off-target effects; and (v) a cell-based delivery system offers the opportunity to add additional functions such as sensing tumour microenvironment via hypoxia sensors, or including suicide switches to improve in vivo control and safety.

In various therapeutic embodiments, the invention provides, among other advantages, a new anti-cancer therapy approach, especially for apoptosis resistant cancer; a method for delivering a protective therapeutic protein for neurodegenerative disease or autoimmune disease, and a method of delivering proteins absent in target cells because of an inherited or acquired genetic variant for metabolic deficiencies and other mendelian diseases.

In some embodiments, the present invention employs a source of lymphocytes that can be engineered, that is, genetically modified, to express a recombinant granzyme fusion protein. For therapeutic uses other than cancer, where the goal may be to deliver, for example, an apoptosis inhibitory protein, then endogenous granzyme and secondary cytotoxic mechanisms may be suppressed. This may be achieved by RNAi, or conventional genome editing technologies such as technologies using zinc finger nucleases (ZFNs), transcription activator-like effector nucleases (TALENs), homing meganucleases, cas9 nucleases (i.e., CRISPR technology), and the like.

The specificity of delivery to desired target cells relies on the specificity of the endogenous antigen receptor on the engineered, or carrier, lymphocyte, or alternatively, in some embodiments, the specificity of a CAR used to co-modify the engineered lymphocyte, or an additional sensing module used to co-modify the engineered lymphocyte.

In some embodiments, a granzyme-toxin fusion protein can be further designed such that the toxin carries an inhibitory domain that is cleaved to activate the toxin payload either within granules or within the cytosol of target cells.

### Sources of Cytotoxic Lymphocytes

In one aspect, cytotoxic lymphocytes are used in methods of the invention. Such cells may be obtained from immortalized cell lines, such as immortalized NK cell lines, for example, NK92, or the like, or they may be obtained from patients or donors (i.e. primary cells) by a variety of techniques known in the art, particularly the art of adoptive cell therapy, e.g. as described in Dudley et al, J. Immunotherapy, 26(4): 332-342 (2003); Dudley et al, Semin Oncol., 34(6): 524-531 (2007).

T lymphocytes can be collected in accordance with known techniques, including known enrichment and depletion techniques, such as discontinuous density gradient centrifugation, magnetic bead affinity separation, fluorescently activated cell sorting (FACS), and the like. After enrichment and/or depletion steps, in vitro expansion of the desired T lymphocytes can be carried out in accordance with known techniques (including but not limited to those described in U.S. Pat. No. 6,040,177 to Riddell et al.), or variations thereof that will be apparent to those skilled in the art.

For example, the desired T cell population or subpopulation may be expanded by adding an initial T lymphocyte population to a culture medium in vitro, and then adding to the culture medium feeder cells, such as non-dividing peripheral blood mononuclear cells (PBMC), (e.g., such that the resulting population of cells contains at least about 5, 10, 20, or 40 or more PBMC feeder cells for each T lymphocyte in the initial population to be expanded); and incubating the culture (e.g. for a time sufficient to expand the numbers of T cells). The order of additional of the T cells and feeder cells to the culture media can be reversed if desired. The culture can typically be incubated under conditions of temperature and the like that are suitable for the growth of T lymphocytes. For the growth of human T lymphocytes, for example, the temperature will generally be at least about 25 degrees Celsius, preferably at least about 30 degrees, more preferably about 37 degrees. The T lymphocytes expanded are typically cytotoxic T lymphocytes (CTL) that are specific for an antigen present on a human tumor or a pathogen. The non-dividing feeder cells can comprise gamma-irradiated PBMC feeder cells. In some embodiments, the PBMC are irradiated with gamma rays in the range of about 3000 to 3600 rads. Optionally, the expansion method may further comprise the step of adding non-dividing EBV-transformed lymphohlastoid cells (LCL) as feeder cells. LCL can be irradiated with gamma rays in the range of about 6000 to 10,000 rads. The LCL feeder cells may be provided in any suitable amount, such as a ratio of LCL feeder cells to initial T lymphocytes of at least about 10:1. Optionally, the expansion method may further comprise the step of adding anti-CD3 monoclonal antibody to the culture medium (e.g., at a concentration of at least about 0.5 ng/ml). Optionally, the expansion method may further comprise the step of adding IL-2 and/or IL-15 to the culture medium (e.g., wherein the concentration of IL-2 is at least about 10 units/ml).

In some embodiments it may be desired to introduce functional genes into the T cells to be used in immunotherapy in accordance with the present invention. For example, the introduced gene or genes may improve the efficacy of therapy by promoting the viability and/or function of transferred T cells; or they may provide a genetic marker to permit selection and/or evaluation of in vivo survival or migration; or they may incorporate functions that improve the safety of immunotherapy, for example, by making the cell susceptible to negative selection in vivo as described by Lupton S. D. et al., Mol. and Cell Biol., 11:6 (1991); and Riddell et al., Human Gene Therapy 3:319-338 (1992); see also the publications of PCT/US91/08442 and PCT/US94/05601 by Lupton et al., describing the use of bifunctional selectable fusion genes derived from fusing a dominant positive selectable marker with a negative selectable marker. This can be carried out in accordance with known techniques (see, e.g., U.S. Pat. No. 6,040,177 to Riddell et al. at columns 14-17) or variations thereof that will be apparent to those skilled in the art based upon the present disclosure.

Engineered tumor infiltrating lymphocytes as cytotoxic lymphocytes: A source of cytotoxic lymphocytes of particular interest are tumor infiltrating lymphocytes (TILs), which may be obtained using conventional techniques, such as described in the Dudley et al (2003). Exemplary techniques are as follows.

Exemplary procedure for excising normal and tumor tissues: Tumor specimens are excised aseptically, and tissue is processed under “good laboratory practice” conditions. “Feeder” lymphocytes as needed are obtained by apheresis of normal donors. All donors are required to undergo testing for infection with common blood-borne pathogens and viruses including RPR, HIV, LCMV, and HVC. Apheresis specimens from normal donors are purified on Ficoll-Hypaque step gradients (LSM Lymphocyte Separation Medium, ICN Biochemicals Inc., Aurora, Ohio) and cryopreserved. Human AB serum may be purchased from several commercial sources (Valley Biomedical, Winchester Va.; Gemini Bioproducts, Woodland, Calif.) after screening for optimal performance to promote the growth of lymphocyte clones.

Exemplary procedure for expanding TIL microcultures: A tumor specimen is dissected free of surrounding normal tissue and necrotic areas. Small chunks of tumor (usually 8-16) measuring about 1 to 2 mm in each dimension are cut with a sharp scalpel from different areas around the tumor specimen. A single tumor fragment is placed in each well of a 24-well tissue culture plate with 2 mL of complete medium (CM) plus 6000 IU per mL of rhIL-2 (Chiron Corp., Emeryville, Calif.). CM consisted of RPMI 1640, 25 mmol/L HEPES pH 7.2, 100 U/mL penicillin, 100 μg/mL streptomycin, 2 mmol/L-glutamine, and 5.5×10⁻⁵ mol/L β-mercaptoethanol, supplemented with 10% human serum. The plates are placed in a humidified 37° C. incubator with 5% CO2 and cultured until lymphocyte growth is evident. Each fragment is inspected about every other day using a low-power inverted microscope to monitor the extrusion and proliferation of lymphocytes. Whether or not lymphocyte growth is visible, half of the medium is replaced in all wells no later than 1 week after culture initiation. Typically, about 1 to 2 weeks after culture initiation, a dense lymphocytic carpet covers a portion of the plate surrounding each fragment. When any well becomes almost confluent, the contents are mixed vigorously, split into two daughter wells and filled to 2 mL per well with CM plus 6000 IU/mL IL-2. Subsequently, the cultures are split to maintain a cell density of 0.8-1.6×10⁶ cells/mL, or half of the media was replaced at least twice weekly.

Exemplary step of obtaining TILs from single cell digests. Briefly, each solid tumor specimen is carefully dissected free of surrounding normal tissue and necrotic areas. The tumor is sliced with a sharp scalpel into small pieces (approximately 2 mm on each side). The tumor fragments are immersed in a mixture of collagenase, hyaluronidase, and DNAse in serum-free RPMI 1640, and incubated overnight with gentle agitation. The single-cell slurry is passed through sterile wire mesh to remove undigested tissue chunks. The digested single-cell suspensions are washed twice in HBSS, viable cells are purified on a single step Ficoll gradient, and cells are resuspended for plating. Multiple wells of a 24-well plate are seeded with 1×10⁶ viable cells in 2 mL CM with 6000 IU/mL IL-2. The plates are placed in a humidified 37° C. incubator with 5% CO2. Whether or not lymphocyte growth is visible, half of the medium is replaced in all wells no later than 1 week after culture initiation. When any well becomes nearly confluent, the contents are mixed vigorously, split into two daughter wells, and filled to 2 mL per well with CM plus 6000 IU/mL IL-2. Subsequently, half the media is replaced at least twice weekly, or the cultures are split to maintain a cell density of 0.8 to 1.6×10⁶ cells/mL. Some of the TIL from digests are derived from multiple original wells that are regularly mixed and eventually pooled for assessment of activity. Other TIL from digests are derived from individual wells of a 24-well plate. For these cultures, all progeny cells from any individual well are treated as an independent TIL culture and are maintained separately from the descendants of any other original well. In this way, multiple cultures may be obtained from the same initial single-cell suspension.

Exemplary method of obtaining TILs by disaggregation: In some embodiments, TILs are derived by a method of physical disaggregation of tumor fragments using a device, such as a Medimachine (Becton Dickenson) with 50 μm “medicon” chambers, which are mini sterile and disposable homogenizers. Fragments of tumor about 2 mm per side may be prepared by dissection of biopsy specimens free from normal and necrotic tissue. Several fragments at a time may be physically disaggregated by a 30-second Medimachine treatment, which disaggregated the tumor chunks using mechanical shear provided by a rotating disk that forced the tumor chunks across a small grater inside the medicon. The resulting slurry of single cells and small cell aggregates is washed once, and resuspended in CM. The cell suspension is layered onto a two-step gradient with a lower step of 100% Ficoll, and a middle step of 75% Ficoll and 25% CM. After 20 minutes' centrifugation at 2000 rpm (about 1100 g), the interfaces are collected. The lower interface containing the lymphocyte-enriched fraction is processed separately from the upper interface containing the tumor-enriched cells. Each fraction is washed twice. The lower, TIL-enriched fraction is plated in 24-well plates, and individual TIL cultures are generated exactly as for the single-cell suspensions derived by enzymatic degradation. The upper, tumor-cell-enriched fraction may be plated at approximately 2×10⁵ cells/mL in RPMI-based media containing 10% “defined” fetal calf serum (Hyclone, Logan, UT) without IL-2.

Exemplary method for assaying TIL activity after isolation: TIL activity and specificity may be determined by analysis of cytokine secretion. TIL and control T-cell lines are washed twice prior to co-culture assay to remove IL-2. TIL cells (1×10⁵) are plated per well of a 96-well flat-bottom plate with 1×10⁵ stimulator cells. TIL cultures are generally stimulated. When available, TILs may be stimulated with an autologous tumor cell line or a thawed aliquot of cryopreserved single-cell tumor digests (“fresh tumor”). For some TILs, the TAP-deficient T2 cell line may be pulsed with tumor antigen peptides (for example, for melanoma: MART-1:27-35 (referred to as MART) or gp100:209-217 (referred to as g209)). After overnight c-culture, supernatants are harvested and IFN-γ secretion is quantified by ELISA (e.g. Pierce/Endogen, Woburn, Mass.).

Exemplary step for expanding engineered cytotoxic lymphocytes for patient infusion: Active TIL cultures may be expanded to treatment levels using a rapid expansion protocol (REP), e.g. described in Riddell et al, Science, 257: 238-241 (1992). Briefly, the REP uses OKT3 (anti-CD3) antibody (Ortho Biotech, Bridgewater, N.J.) and IL-2 in the presence of irradiated, allogeneic feeder cells at a 200:1 ratio of feeder cells to responding TIL cells. PBMC feeder cells obtained from normal volunteers by apheresis are thawed, washed, resuspended in CM, and irradiated (50 Gy). PBMC (2×10⁸), OKT3 antibody (30 ng/mL), CM (75 mL), AIM V media (GIBCO/BRL, 75 mL), and TIL effector cells (1×10⁶) are combined, mixed, and aliquoted to a 175 cm2 tissue culture flask. Flasks are incubated upright at 37° C. in 5% CO2. IL-2 is added to 6000 IU/mL on day 2. On day 5, 120 mL of culture supernatant is removed by aspiration (cells are retained on the bottom of the flask) and media was replaced with a 1:1 mixture of CM/AIM V containing 6000 IU/mL IL-2. On day 6 and every day thereafter, cell concentration is determined and cells are split into additional flasks or transferred to Baxter 3-L culture bags with additional medium containing 6000 CU/mL IL-2 as needed to maintain cell densities around 1×10⁶ cells/mL. About 14 days after initiation of the REP, cells are harvested from culture bags and prepared for patient treatment. Harvesting is accomplished using a Baxter/Fenwal continuous centrifuge cell harvester system. The cells are then washed in 0.9% sodium chloride and resuspended in 45 to 150 mL of 0.9% sodium chloride with 2.5% human albumin Samples are removed from the infusion product for QC testing, aliquots are cryopreserved for future experimental analysis, and the remaining cells are infused into the patient by intravenous administration over 30 minutes.

### Engineering Lymphocytes

In accordance with the invention, cells are engineered to couple a cellular recognition system (such as, T cell receptors, CARs, or the like) with granzyme-perforin systems to provide cell-based compound delivery to targeted cell populations. Whenever such cells are cytotoxic lymphocytes they may be referred to herein as “delivery lymphocytes”, or “engineered cytotoxic lymphocytes,” or like terms. In some embodiments, such engineered cells are lymphocytes that already possess one or both of these capabilities, which may be modified using conventional genetic engineering techniques to permit delivery of predetermined compounds, such as predetermined proteins. In particular, in some embodiments of the invention, cytotoxic lymphocytes are genetically modified so that they produce a fusion protein comprising an effector agent and a predetermined protein, which is sequestered in a lytic granule. In some embodiments, such fusion proteins sequestered in lytic granules are released in response to specific recognition of an MHC-epitope complex on a target cell by a receptor of the engineered cytotoxic lymphocyte. In other embodiments, specific recognition of a target cell may be accomplished by cytotoxic lymphocytes expressing a CAR specific for a surface antigen of the target cell.

The above genetic modifications may be carried out using conventional mammalian cell genetic engineering techniques, including plasmid or RNA transfection, transduction by viral vectors and direct genome editing using programmable nucleases, such as CRISPR systems, TALENs, zinc finger nucleases, and the like. Guidance for applying such techniques to the present invention may be found in the following references, which are incorporated herein by reference: Kim et al, Nature Reviews Genetics, 15: 321-334 (2014); Gaj et al, Trends Biotechnology, 31(7): 397-405 (2013); Hsu et al, Cell, 157: 1262-1278 (2014); Sander et al, Nature Biotechnology, 32(4): 347-355 (2014); June et al, Nature Reviews Immunology, 9: 704-716 (2009); Schmidt et al, Biotechnology J., 10: 258-272 (2015); Senis et al, Biotechnology Journal, 9: 1402-1412 (2014)(including supplemental materials); and like references.

In some embodiments, genetic modification is made by transducing cytotoxic lymphocytes using a vector that stably integrates into its genome. Exemplary vectors for such transduction includes, but is not limited to, lentivirus and retrovirus vectors, adenoviruses, adeno-associated virus (AAV), transposons, and the like. In some embodiments, retroviral vectors are used to transduce cytotoxic lymphocytes, e.g. as taught by Halene et al, Blood, 94: 3349-3357 (1999), which is incorporated by reference. In other embodiments, a lentivirus vector is employed to transduce cytotoxic lymphocytes. Lentivirus vectors that can readily be modified to incorporate fusion proteins are commercially available, for example, from Takara-Clontech (Mountain View, Calif.), Cellecta (Mountain View, Calif.), and like vendors. Guidance in modifying and using lentivirus vectors is provided in the following references, which are incorporated herein by reference: Liechtenstein et al, Cancers, 5: 815-837 (2013); Dornmair et al, U.S. patent publication 2013/0195900; Breckpot et al Gene Therapy, 14: 847-862 (2007); Wong-Staal et al, U.S. patent publication 2001/0007659; Bauche et al, U.S. patent publication 2014/0120132; and the like.

In accordance with some embodiments of the invention, methods of treating an individual with an apoptotic resistant disease, such as a cancer, include steps of obtaining cytotoxic lymphocytes from an individual, genetically modifying the isolated cytotoxic lymphocytes so that they are capable of producing lytic granules that sequester a fusion protein comprising an effector agent and a toxin, expanding the genetically modified cytotoxic lymphocytes to generate therapeutic amounts of cells, and administering to the individual a therapeutically effective amount of the genetically modified cytotoxic lymphocytes. In some embodiments, cytotoxic lymphocytes are genetically modified by transducing the cytotoxic lymphocytes using a viral vector.

In some embodiments it may be useful to include in the delivery cells a positive marker that enables the selection of cells of the negative selectable phenotype in vitro. The positive selectable marker may be a gene which, upon being introduced into the host cell expresses a dominant phenotype permitting positive selection of cells carrying the gene. Genes of this type are known in the art, and include, inter alia, hygromycin-B phosphotransferase gene (hph) which confers resistance to hygromycin B, the aminoglycoside phosphotransferase gene (neo or aph) from Tn5 which codes for resistance to the antibiotic G418, the dihydrofolate reductase (DHFR) gene, the adenosine deaminase gene (ADA), and the multi-drug resistance (MDR) gene. Resistance markers, such as the foregoing, are useful when transfecting a plasmid into cytotoxic lymphocytes. Plasmids are episomal and do not replicated, and are eventually lost or diluted out by cell division. By using a selective marker, like the above, one may select for the cells where there is a rare genomic integration event of the plasmid. For viral transduction, such as with lentivirus, there is routine genome integration, so selection using a resistance marker is unnecessary. Instead, a fluorescent protein driven by an appropriate mammalian promoter may be included in the virus, such that when the virus integrates, the fluorescent protein will be expressed as a marker to sort or otherwise follow the transduced cell population. For example, in some embodiments, a gene for a fluorescent protein, such as green fluorescent protein (GFP), may be inserted in a multi-cystronic viral construct, usually in the C-terminal or 3′ position.

Preferably, the positive selectable marker and the negative selectable element are linked such that loss of the negative selectable element necessarily also is accompanied by loss of the positive selectable marker. Even more preferably, the positive and negative selectable markers are fused so that loss of one obligatorily leads to loss of the other. An example of a fused polynucleotide that yields as an expression product a polypeptide that confers both the desired positive and negative selection features described above is a hygromycin phosphotransferase thymidine kinase fusion gene (HyTK). Expression of this gene yields a polypeptide that confers hygromycin B resistance for positive selection in vitro, and ganciclovir sensitivity for negative selection in vivo. See Lupton S. D., et al, Mol. and Cell. Biology 11:3374-3378, 1991. In addition, in preferred embodiments, the polynucleotides of the invention encoding the chimeric receptors are in retroviral vectors containing the fused gene, particularly those that confer hygromycin B resistance for positive selection in vitro, and ganciclovir sensitivity for negative selection in vivo, for example the HyTK retroviral vector described in Lupton, S. D. et al. (1991), supra. See also the publications of PCT/US91/08442 and PCT/US94/05601, by S. D. Lupton, describing the use of bifunctional selectable fusion genes derived from fusing a dominant positive selectable markers with negative selectable markers.

Preferred positive selectable markers are derived from genes selected from the group consisting of hph, neo, and gpt, and preferred negative selectable markers are derived from genes selected from the group consisting of cytosine deaminase, HSV-I TK, VZV TK, HPRT, APRT and gpt. Especially preferred markers are bifunctional selectable fusion genes wherein the positive selectable marker is derived from hph or neo, and the negative selectable marker is derived from cytosine deaminase or a TK gene.

A variety of methods can be employed for transducing lymphocytes, as is well known in the art. For example, retroviral transductions can be carried out as follows: on day 1 after stimulation using REM (as described in U.S. Pat. No. 6,040,177; which is incorporated herein by reference), provide the cells with 20-30 units/ml IL-2; on day 3, replace one half of the medium with retroviral supernatant prepared according to standard methods and then supplement the cultures with 5 ug/ml polybrene and 20-30 units/ml IL-2; on day 4, wash the cells and place them in fresh culture medium supplemented with 20-30 units/ml IL-2; on day 5, repeat the exposure to retrovirus; on day 6, place the cells in selective medium (containing, e.g., an antibiotic corresponding to an antiobiotic resistance gene provided in the retroviral vector) supplemented with 30 units/ml IL-2; on day 13, separate viable cells from dead cells using Ficoll Hypaque density gradient separation and then subclone the viable cells.

In some embodiments, transgenes may be introduced for expression in cytotoxic lymphocytes by in vitro RNA transfection, for example, as disclosed by Coughlin et al, Blood, 103(6): 2046-2053 (2004), which is incorporated herein by reference. Briefly, for example, RNA capable of expressing a fusion protein of interest is transcribed in vitro, and then electroporated into the desired cells. After uptake, the target cells express the RNA to produce the fusion protein of interest.

In some embodiments, as mentioned above, lentiviruses may be used to insert fusion proteins and other transgenes into delivery lymphocytes. Lentiviral vectors can mediate the efficient delivery, integration and stable or controlled expression of transgenes in dividing as well as non-dividing cells in vitro. Kits and materials for constructing lentivirus vectors are available commercially, e.g. Addgene (Cambridge, Mass.), and guidance for their application is found in the following exemplary references: Dull et al, J. Virol., 72(11): 8463-8471 (1998); Naldini et al, Science, 272(5259): 263-267 (1996); and the like. Briefly, a common protocol calls for construction of at least three precursor vectors: a transfer vector containing a transgene, an envelope vector and a packaging vector. The three vectors are co-transfected into a virus production cell line, such as A293T cells, which serve as a production host for packaged lentivirus. The packaged lentivirus is harvested from the A293T culture and adjusted to a titer for infecting target cytotoxic lymphocytes.

In some embodiments, cytotoxic lymphocytes may be engineered using a CRISPR system to express fusion proteins for delivery target cells. With this approach, a fusion protein may be created by directly modifying the granzyme genomic locus in NK or T cells by using, for example, a CRISPR system, or a like system (such as Zinc Finger Nucleases or TALENs) for direct genomic modification. In some embodiments, such modification may be carried out in vitro or in vivo, where the transgene (for example, encoding a fusion protein) and cas9 gene and guide RNAs are delivered by a viral vector like AAV, such as described in Senis et al, Biotechnology Journal, 9: 1402-1412 (2014)(including supplemental materials), which are incorporated herein by reference. Such an approach is advantageous because the transgene would be under the control of the native granzyme promoter and other native regulatory elements, enhancers, and so on, which may be important for optimal expression and loading into lytic granules. Thus, possible inefficiencies may be reduced which are due to competition by the native granzyme for vesicle loading with granzyme fusion proteins from transfection or transduction.

In some embodiments, cytotoxic lymphocytes may be genetically modified by the following steps: (i) transfecting cytotoxic lymphocytes for constitutive or transient expression transgenes encoding a programmable nuclease; (ii) transfecting the cytotoxic lymphocytes with donor templates for homology-directed repair of double stranded breaks produced by the template-specific nuclease, the donor templates encoding a fusion protein comprising an effector agent and a predetermined protein and comprising sequences to target the donor template to the double stranded break produced by the template-specific nuclease. In some embodiments, a programmable nuclease is a ZFN, TALEN, homing meganuclease, or an RNA-guided nuclease (RGN). In some such embodiments, an RGN is a component of a CRISPR system. In some embodiments, the RGN is a Cas9 nuclease. In some embodiments, the effector agent is a granzyme. In some embodiments, the predetermined protein is a toxin. In some embodiments, the donor template is a component of a double stranded plasmid. In some embodiments the donor template is a component of a viral vector. In some embodiments, the step of transfecting cytotoxic lymphocytes with an RGN and guide RNA results in transient expression thereof. In some embodiments, the step of transfecting cytotoxic lymphocytes with an RGN and guide RNA is carried out with a viral vector. In some embodiments, such viral vector may be a lentivirus, AAV, gammaretrovirus, spumavirus, Ad5-35, lymphotropic herpesvirus, or the like.

In some embodiments, an effector agent, such as granzyme B, is fused to the Cre enzyme to produce fusion protein (“GzB-Cre”), which is useful for detecting on target and off target delivery with cells or tissues or experimental animals that carry a floxed reporter transgene. An exemplary nucleotide sequence encoding a GzB-Cre fusion protein is listed as SEQ ID NO: 3.

### Delivery of Nucleic Acids and Small Molecules

In some embodiments, the invention provides cell-based delivery of compounds, which may include nucleic acids and small molecules, such as drugs, dyes, organic molecules, or the like, as well as proteins. In some embodiments, this aspect of the invention may be implemented using specific nucleic acid sequences, e.g. aptamers, which have been designed and selected to bind specific amino acid sequences or specific protein domains, or specific small molecules, or some combination thereof. For example, such a nucleic acid-binding- or small molecule-binding protein or peptide may be fused to a granzyme. Then, for nucleic acid delivery, a desired nucleic acid may be added to the fusion protein so that the nucleic-acid-binding portion of the protein captures the desired nucleic acid. In some embodiments, the captured nucleic acid may be a single nucleic acid, that has both a fusion protein binding domain and a separate domain conferring a desired functionality, either due to intrinsic effects of the nucleic acid sequence itself, or alternatively small molecule binding, providing a structural linker functionality. For the nucleic acid binding protein domain, two options include using a standard protein tag (e.g. MS2 which has known nucleic acid aptamers that binds to it), or a more complex system such as a bacterial P1 system, which might be more useful for whole plasmid delivery, e.g. Schumacher et al, Nature, 438(24): 516-519 (2005). For small molecule delivery, the nucleic acid becomes a linker, with one region binding to the protein domain fused to granzyme, and the other region binding to a small molecule. Such bispecific aptamers may be made using conventional techniques, e.g. SELEX, disclosed in U.S. Pat. No. 5,580,737. In some embodiments, such small molecule delivery may be used for pro-cell health molecules or stimulatory molecules, rather than toxic ones, such as dopamine.

As used herein, “small molecule” means a molecule having a molecular weight of less than 1000 daltons. In some embodiments, a small molecule comprises an organic molecule. In other embodiments, a small molecule comprises a drug.

### Therapeutic Compositions

In accordance with the invention, a wide range of one or more predetermined proteins may be fused with one or more effector agents or portions thereof for specific delivery to a population of target cells whether for therapeutic purposes, or for other purposes. In some embodiments, predetermined proteins have a therapeutic function, such as, target cell killing when the invention is employed as an anti-cancer therapeutic. Selection of predetermined proteins is limited only by what can be accommodated by the granzyme-perforin pathway. For example, the total size of a fusion protein is limited by the size of perforin pores through which molecule fusion protein is delivered to target cell cytoplasm. This is approximately 20 nm. Fusion proteins comprising granzyme B plus any of a number of toxins (e.g. diptheria toxin, pseudomonas toxin, saporin) or anti-apoptotic proteins (e.g. XIAP) are within this limit. In some embodiments, predetermined proteins include mammalian pro-apoptotic proteins; that is, proteins that promote apoptosis. Mammalian pro-apoptotic proteins include, but are not limited to, hBAX, caspases, DIABLO, DNAse and the like. In other embodiments, predetermined proteins include, but are not limited to, bacterial toxins such as DT, PE, BarRNAse; and plant toxins such as ricin and saporin. In some embodiments, a predetermined protein is selected from the group consisting of diphtheria toxin, pseudomonas toxin, thymidine kinase, and BaRNAse.

Predetermined protein toxins for use in the invention may include toxins used in immunotoxin therapy, e.g. Pastan et al, Nature Reviews Cancer, 6:559-565 (2006), which is incorporated herein by reference. In some embodiments, toxins for use in the invention include Pseudomonas aeruginosa exotoxin A (PE), diphtheria toxin (DT), ricin, saporin or fragments thereof. In some embodiments, factors that may be used for selecting toxins for use in the invention include: (i) they are genetically encodable; (ii) they are relatively small, i.e. a toxin-effector agent fusion protein can traverse a perforin pore; (iii) they act in the cytosol of targeted cells; and (iv) fusion proteins with toxin activity can be constructed. In other embodiments, other factor may be applicable, for example, if a predetermined protein includes a nuclear localization sequence, or the like.

In some embodiments, predetermined proteins may be therapeutic proteins that provide a therapeutic effect indirectly by rendering target cells susceptible to treatment by other drugs. Such protein may alter membrane permeability, receptor function, or other cellular functions affecting susceptibility to treatment. In some embodiments, such indirectly acting therapeutic protein may provide a target for predetermined drugs. For example, a fusion of GzB to thymidine kinase (TK) renders target cells susceptible to killing by gancyclovir (e.g. SEQ ID NO: 4). In such embodiment, TK is delivered specifically to tumor cells of a patient with the method of the invention, after which the patient is dosed with gancyclovir. In some embodiments, to avoid adversely affecting delivery cells, administration of the TK-specific drug would occur subsequently to delivery of TK molecules.

In embodiments where a delivery lymphocyte delivers a beneficial compound to a target cell population, the delivery lymphocyte's cytotoxic functions are eliminated or attenuated so that target cells are not damaged or destroyed during contact with a delivery lymphocyte. For example, in embodiments where a cytotoxic lymphocyte is used to deliver a predetermined protein to repair a disease state in a cell (e.g. XIAP reversing cell death in an autoimmune disorder, a missing enzyme inborn error of metabolism, dopamine in Parkinson's, or the like) the endogenous cytotoxic activity of the delivery cytotoxic lymphocyte is clearly at odds with the therapy; thus, in such in embodiments the cytotoxic functions of the delivery cells are genetically turned off, or otherwise attenuated. In some embodiments, the effector agent (e.g. granzyme B) portion of a fusion protein used to deliver the payload may be inactivated. This can be achieved by either mutating the full length protein (in granzyme's case this involves making 1-2 amino acid substitutions in the catalytic site of granzyme) to make it incapable of killing the target cell, or using a portion of the effector agent without cytotoxic functionality. Likewise, in embodiments designed to deliver beneficial compounds to target cells, all of the endogenous effector mechanisms of the delivery lymphocytes are ablated or attenuated. For example, many such effector mechanisms may be attenuated to low levels using RNA interference (microRNA, antisense, etc) to prevent gene expression, or they could be knocked out entirely using programmable nucleases (ZFNs, CRISPR etc).

### Therapeutic Applications

Subjects that can be treated by the present invention are, in general, mammals, particularly primates, and most particularly humans. Human subjects can be male or female and can be any suitable age, including infant, juvenile, adolescent, adult, and geriatric subjects.

Subjects that can be treated include subjects afflicted with cancer, including but not limited to colon, lung, liver, breast, prostate, ovarian, skin (including melanoma), bone, and brain cancer, etc. In some embodiments the tumor associated antigens are known, such as melanoma, breast cancer, squamous cell carcinoma, colon cancer, leukemia, myeloma, prostate cancer, etc. (in these embodiments memory T cells may be isolated or engineered by introducing the T cell receptor genes). In other embodiments the tumor associated proteins can be targeted with genetically modified T cells expressing an engineered immune receptor. Examples include but are not limited to B cell lymphoma, breast cancer, prostate cancer, and leukemia.

Subjects that can be treated also include subjects afflicted with, or at risk of developing, an infectious disease, including but not limited to viral, retroviral, bacterial, and protozoal infections, etc.

Subjects that can be treated include immunodeficient patients afflicted with a viral infection, including but not limited to Cytomegalovinis (CMV), Epstein-Barr virus (EBV), adenovirus, BK polyomavirus infections in transplant patients, etc.

Subjects that can be treated further include individuals suffering from single gene disorders (for example, enzymatic deficiencies); and autoimmune and degenerative disorders (MS, ALS, rheumatoid arthritis).

Cells prepared as described above can be utilized in methods and compositions for adoptive immunotherapy in accordance with known techniques, or variations thereof that will be apparent to those skilled in the art based on the instant disclosure. See, e.g., U.S. Patent Application Publication No. 2003/0170238 to Gruenberg et al; see also U.S. Pat. No. 4,690,915 to Rosenberg.

In some embodiments, the cells are formulated by first harvesting them from their culture medium, and then washing and concentrating the cells in a medium and container system suitable for administration (a “pharmaceutically acceptable” carrier) in a treatment-effective amount. Suitable infusion medium can be any isotonic medium formulation, typically normal saline, Normosol R (Abbott) or Plasma-Lyte A (Baxter), but also 5% dextrose in water or Ringer's lactate can be utilized. The infusion medium can be supplemented with human serum albumen.

A treatment-effective amount of cells in an administered composition can be any number sufficient to allow expansion after administration. Such number may vary widely based on the source of cytotoxic lymphocytes that are engineered, the type of genetic modification used (for example, transfection, transformation, direct gene editing, e.g. using CRISPR, or like system), the type of ex vivo culturing techniques used, and the like. In some embodiments, treatment-effective amount of cells in the composition is at least 10³, 10⁴, 10⁵, 10⁶, 10⁷, 10⁸, or 10⁹ cells. In some embodiments, a treatment-effective amount of cells is typically at least 10⁶, 10⁷, 10⁸, or 10¹⁰ cells. The number of cells will depend upon the ultimate use for which the composition is intended as will the type of cells included therein. For example, if cells that are specific for a particular antigen are desired, then preferably the population will contain a greater percentage of such cells. In some embodiments, a composition includes at least 10% target specific genetically modified cells; in other embodiments, such composition contains at least 25% of such cells; in still other embodiments, such composition contains at least 50% of such cells. For uses provided herein, the cells are generally in a volume of a liter or less, can be 500 mls or less, even 250 mls or 100 mls or less. Hence the density of the desired cells is typically greater than 10⁶ cells/ml and generally is greater than 10⁷ cells/ml, generally 10⁸ cells/ml or greater. The clinically relevant number of immune cells can be apportioned into multiple infusions that cumulatively equal or exceed 10⁹, 10¹⁰ or 10¹¹ cells.

In some embodiments, the lymphocytes of the invention may be used to confer immunity to individuals. By “immunity” is meant a lessening of one or more physical symptoms associated with a response to infection by a pathogen, or to a tumor, to which the lymphocyte response is directed. Lymphocytes of the invention may include cytotoxic lymphocytes or memory lymphocytes. The amount of cells administered is usually in the range present in normal individuals with immunity to the pathogen. Thus, in some embodiments, the cells are usually administered by infusion, with each infusion in a range of at least 10⁶ to 10¹⁰ cells/m2, preferably in the range of at least 10⁷ to 10⁹ cells/m2. The clones may be administered by a single infusion, or by multiple infusions over a range of time. However, since different individuals are expected to vary in responsiveness, the type and amount of cells infused, as well as the number of infusions and the time range over which multiple infusions are given are determined by the attending physician, and can be determined by routine examination. The generation of sufficient levels of T lymphocytes (including cytotoxic T lymphocytes and/or helper T lymphocytes) is readily achievable using the rapid expansion method of the present invention, as exemplified herein. See, e.g., U.S. Pat. No. 6,040,177 to Riddell et al. at column 17.

### Example 1

### Functional Validation of Granzyme Fusion Proteins

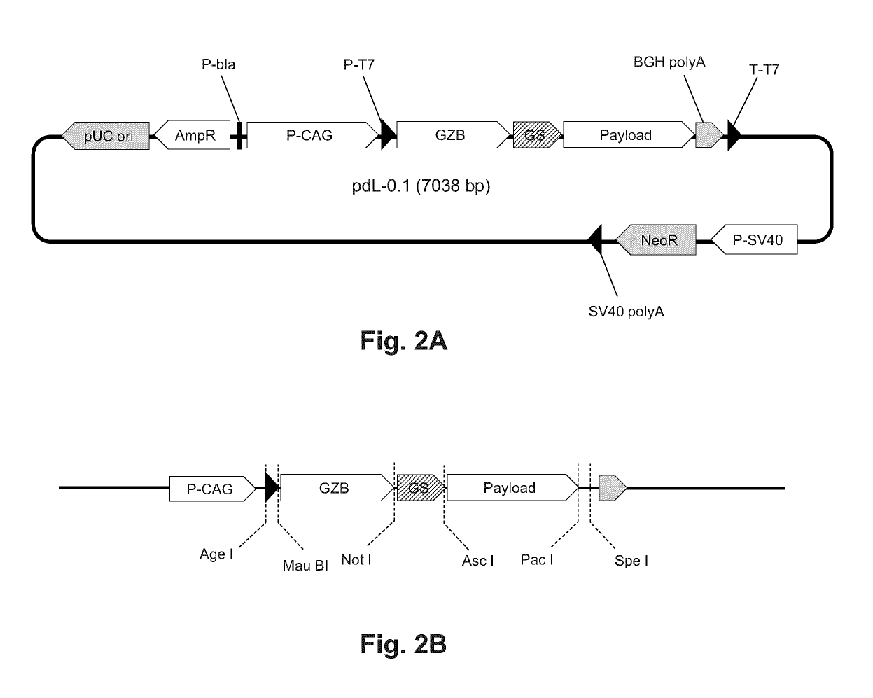

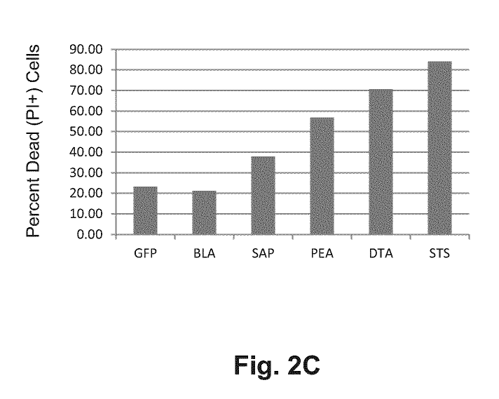

HeLa cells were transfected using Lipofectamine LTX and PLUS reagent (Life Technologies) with pdL plasmids (constructs shown diagrammatically in FIGS. 2A and 2B) expressing green fluorescent protein (GFP), GzB-BLA (β-lactamase, a reporter), GzB-SAP (Saporin, a plant toxin), GzB-PEA (pseudomonas exotoxin A, a bacterial toxin) and GzB-DTA (diphtheria toxin fragment A, a bacterial toxin), and incubated for 48 h. FIG. 2B shows the locations of restriction endonuclease recognition sites used in assembling elements of the vector. Exemplary nucleotide sequences encoding GzB-DTA and GzB-SAP are provided as SEQ ID NO: 1 and SEQ ID: 2, respectively. Positive and negative controls consisted of 24 h treatment with 1 uM staurosporine (a small molecule kinase inhibitor, STS) and vehicle only respectively. Cells were trypsinized, stained with propidium iodide (PI), and dead (PI+) cells quantified by flow cytometry. These values were corrected for transfection efficiency. Given that the payloads are fused to full-length inactive pro-granzyme, and that the granzyme-toxin fusions have markedly higher toxicity than the GFP-only GzB-BLA controls, these results demonstrate that the toxin payloads effectively mediate GzB independent cell death. Results are shown in FIG. 2C.

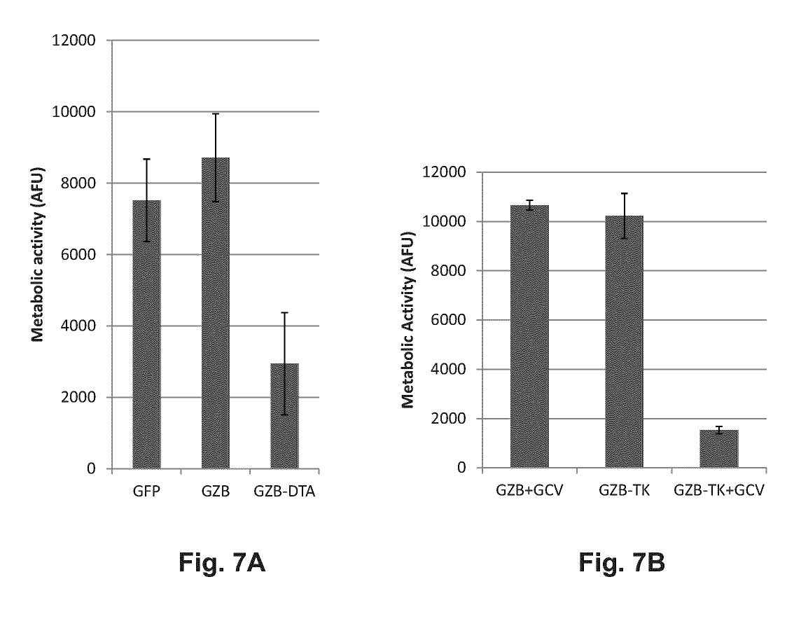

Another demonstration of the efficacy of the granzyme-toxin fusion proteins is shown in FIG. 7A and 7B. MND vectors with either GFP (as a negative control), granzyme alone (GZB), granzyme fused to thymidine kinase (GZB-TK) or granzyme fused to diphtheria toxin (DTA) were transfected into HEK 293T cells. 24 hours after transfection cells were treated with ganciclovir (GCV) where appropriate. 48 hours after transfection, cells were harvested, diluted and reseeded. 72 hours after reseeding, cells were harvested, and cell viability was quantified using a resazurin-based fluorescent metabolic activity assay (PrestoBlue). These experiments were conducted in triplicate, with the results shown in FIG. 7A and 7B. Error bars represent standard deviation. GZB-TK and granzyme with GCV showed no toxic effects. However when these two components were combined GZB-TK activated ganciclovir to exert a potent toxic effect, as expected. Similarly, GZB-DTA showed potent toxicity compared to GZB alone, and the GFP negative control.

### Example 2

Vectors for Production and Transfer of Granzyme B Fusion Proteins to Target Cells

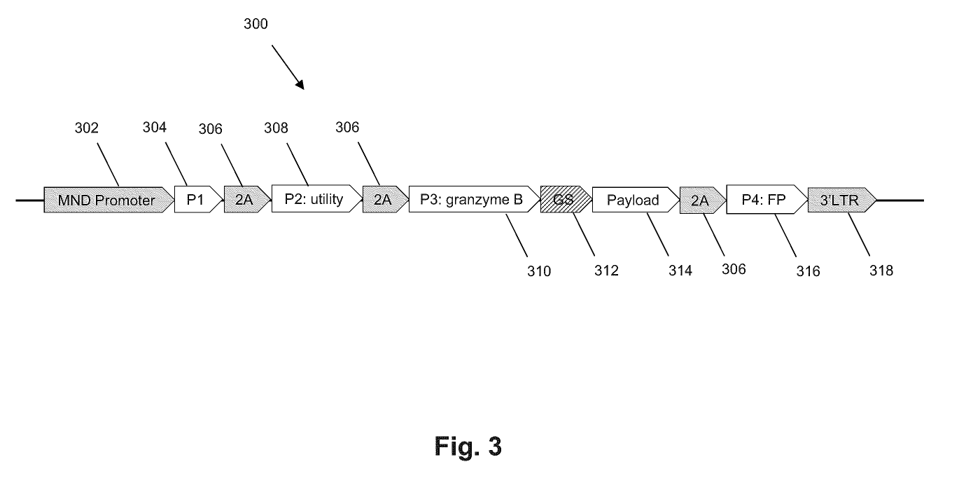

A modular polycistronic viral vector has been designed for modifying NK cells based on a third generation self-inactivating lentiviral vector (300), as illustrated in FIG. 3, and as disclosed in Okita et al, Nature Protocols, 5: 418-28 (2010), which is incorporated herein by reference. MND promoter (302) drives robust expression of a four-component polyprotein across a variety of tissues. Each component P1 (304), P2 (308), P3 (310, 312, 314), and P4 (316) is separated by an orthogonal 2A sequence (306) that self-cleaves upon folding to release each individual protein. In some embodiments, P1 (304) may be used to encode a chimeric antigen receptor, while the P2 (308) “utility” position may be used for additional cell modifications, such as expression of mutant EF2 to protect host cells from deleterious effects of a payload protein, e.g. diphtheria toxin (DTA) toxicity. P3 (310, 312, 314) consists of a GzB (310)-Payload (314) fusion protein. Payloads consisting of tdTomato and DTA have been tested. Finally, the C-terminal P4 position (318) contains a fluorescent tag (GFP or CFP), which enables enrichment and tracking of the transduced cell population. Its position at the C-terminal also ensures that if a fluorescent signal is observed, all other component proteins are in-frame and expressed. Finally, each module depicted is flanked by unique restriction enzyme cleavage sites (not shown in FIG. 3) to facilitate rapid modification.

Co-culture experiments to demonstrate targeted delivery of therapeutic proteins using engineered cytotoxic lymphocytes. NK-92 cells are virally transduced to express GzB-Payload fusion proteins that are loaded into lytic granules. After FACS enrichment using the fluorescent tag (316, FIG. 3) the GzB-Payload expressing NK-92 cells are co-cultured with target K562 cells. Upon NK cell engagement of target cells, the granzyme fusion proteins are released into the immunological synapse between the NK and K562 cells, and perforin pores facilitate fusion protein access to the target cell, whereupon the payload exerts its therapeutic effect.

### Example 3

### Transduction of NK Cells and Production and Transfer of Granzyme B Fusion Proteins to Target Cells

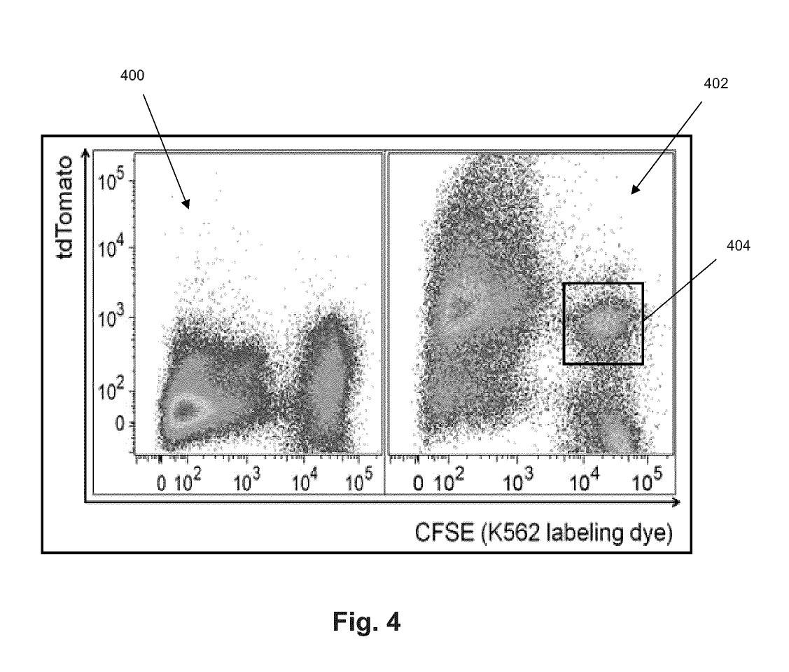

In this example GzB-tdTomato fusion proteins from NK cells were transferred to target K562 cells. NK cells were electroporated with a plasmid encoding a GzB-tdTomato (GzB-tdT) fusion protein, and then enriched for fusion protein expressing cells (dL-tdTs) via FACS sorting of tdT+NK cells. These dL-tdTs were co-cultured with CFSE labeled K562s at a 4:1 effector:target ratio for 4 hours, and then analyzed on a flow cytometer(402 in FIG. 4). The presence of the CFSE+tdT+K562 population (enclosed in black box (404) on the right panel), and the absence of this population in the untransfected NK/K562 co-culture (shown in left pane(400)), indicates successful transfer of the tdT payload from NK cell to target K562 cell.

### Example 4

Model for Apoptosis Resistant Cells

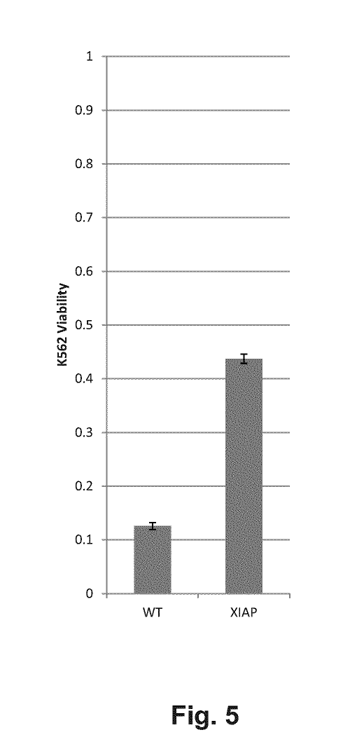

In this example, a model is constructed for cells resistant to apoptosis to test embodiments of the invention. Target cells are produced that overexpress the inhibitor of apoptosis proteins (IAPs) survivin and/or X-linked Inhibitor of Apoptosis Protein (XIAP). Overexpression of IAP family members blocks the activation of executioner caspases 3 and 7 by granzyme or other intrinsic activators, and IAP up-regulation is both a natural apoptosis resistance mechanism of tumors and an established method for preventing apoptosis experimentally. Results are shown in FIG. 5.

Inhibition of NK-92 killing of target K562s. Data in FIG. 5 shows XIAP overexpression renders K562 cells resistant to NK killing. K562 cells were transduced with an MND vector coding for a polyprotein consisting of the X-linked inhibitor of apoptosis protein (XIAP) in P3 and cerulean fluorescent protein (CFP) in P4. CFP+cells were enriched to purity using FACS sorting, and then co-cultured with NK-92MI cells for 4 hours at a 4:1 effector to target ratio. The co-cultures were stained with propidium iodide (PI) and dead (PI+) cells were quantified via flow cytometry. Therefore, these results indicate that XIAP overexpression render K562s very resistant to NK attack.

### Example 5

### Protection of Delivery Cells From DT Toxicity

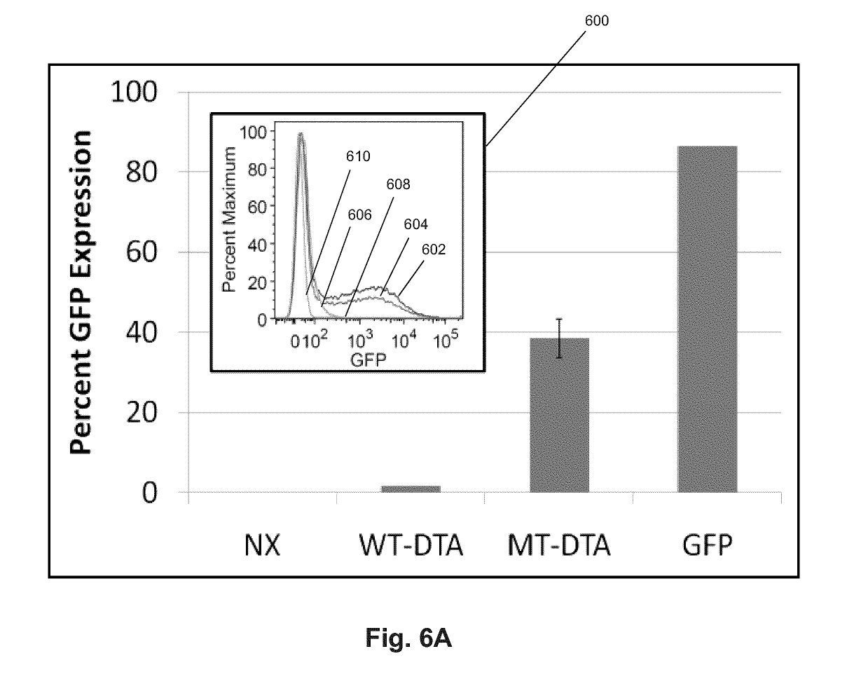

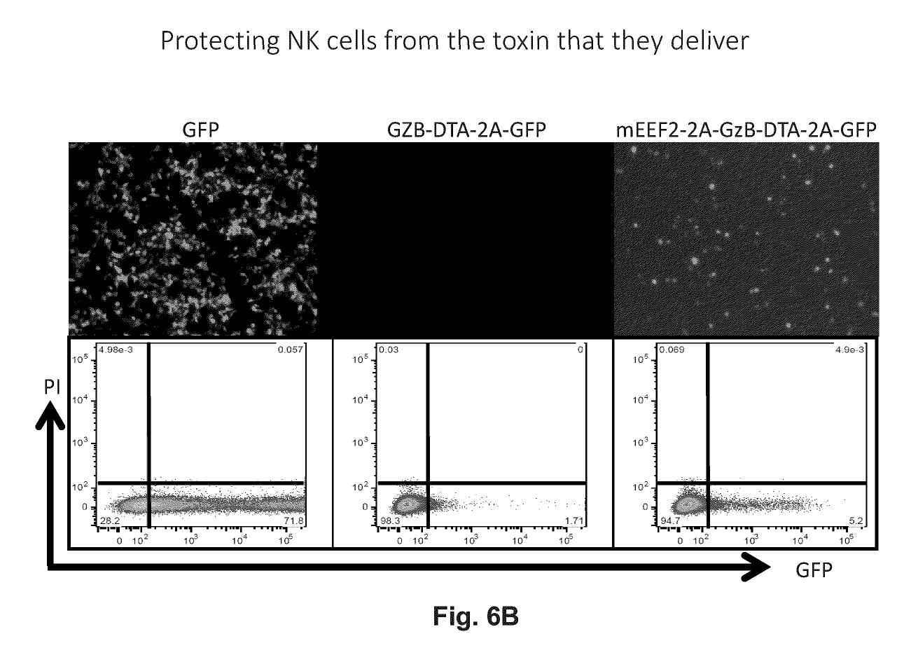

In this example, a method to protect delivery cells from diphtheria toxicity was developed. Diptheria toxin (DT) should be highly effective in doses that can be delivered by a lymphocyte, as the amount of toxin delivered should be similar to the amount of physiological GzB delivered, and DT is more potent than GzB. Further, DT kills by inhibiting elongation factor 2 (EF2), which blocks target cell protein synthesis, a cytotoxic mechanism entirely independent of conventional lymphocyte cytotoxicity pathways, and one that does not depend on apoptosis induction. Furthermore it is a completely ‘orthogonal’ toxin, in the sense that, unlike all other immune effector mechanisms, the tumor cells have not evolved under the selective pressure of DT, and hence are completely naïve to its effects. To ensure delivery cell viability when carrying GzB-DT, a mutant version of EF2 has been generated with the amino acid substitution G717R, e.g. Wang et al, Gene Therapy, 17: 1063-1076 (2010). Overexpression of EF2 with this mutation (mEF2) has been shown to protect cells from DT toxicity (FIGS. 6A and 6B). In some embodiments, a gene encoding a toxin protection factor may be incorporated into a delivery lymphocyte. In this example, mEF2 is incorporated into a lentiviral construct that encodes the GzB-DT fusion protein. Transduction of NK cells with this construct will allow simultaneously the expression of GzB-DT and the protection of NK cells from DT toxicity.

Mutant EF2 (mEF2) overexpressed in HEK293T cells rescues protein expression from Diphtheria Toxin blockade. Wild type (WT) or mEF2 expressing (MT) HEK293T cells were transfected with a MND vector encoding a polyprotein consisting of the GzB-DTA fusion protein in P3 and GFP in P4. 48 hours after transfection, the cells were analyzed for GFP expression via flow cytometry. The inset (600) of FIG. 6A shows a histogram of GFP expression for MT (blue (602) and red (604)), WT (orange(606) and turquoise(608)) duplicates, as well as an untransfected negative control (green (610)). The histogram and the bar graph clearly demonstrate that in the presence of GzB-DTA, unmodified HEK293Ts (WT) produce virtually no GFP, while in mEF2 expressing HEK293Ts (MT), robust GFP expression is observed in the presence of GzB-DTA. NX is an untransfected negative control and GFP alone is used as a positive control. Both controls are single experiments. Duplicate experiments were conducted for WT and MT, reported as Mean +/−SD (error bars are negligible for WT-DTA).

In a separate experiment, HEK293Ts were transfected with plasmids encoding for polyproteins (from left to right in FIG. 6B): GFP, GZB-DTA_2A_GFP, and mEEF2_2A_GZB-DTA_2A_GFP. 48 hours after transfection the GFP fluorescent intensity was imaged using a fluorescence microscope (top panels) and then quantified by flow cytometry (bottom panels). The complete elimination of the GFP signal in the middle panel (GZB-DTA_2A_GFP) indicates strong inhibition of protein synthesis. The partial restoration of this signal in the right panel (mEEF2_2A_GZB-DTA_2A_GFP) indicates that mEEF2 is providing sufficient protein synthesis capacity to enable production of GFP, and thus that mEEF2 co-expressed with DTA protects cells from DTA mediated inhibition of protein synthesis.

### Example 6

### Treating Apoptosis Resistant Tumor in a Mouse Model

In this example, GzB-DTA engineered lymphocytes are characterized in vivo using a mouse model system. The response is measured of implanted IAP-overexpressing lymphocyte-resistant tumors to adoptively transferred tumor-specific cytotoxic T lymphocytes that express the GzB-DTA fusion protein.

To test anti-tumor activity in vivo, cytotoxic lymphocytes with tumor antigen specificity are used. The effector cells used are primary CD8+ splenocytes from OT-I transgenic mice, which express an invariant TCR specific for the SIINFEKL peptide epitope of the model antigen ovalbumin (OVA). The immortalized mouse lymphoblast cell line EG.7-OVA constitutively expresses the OVA transgene and presents the SIINFEKL epitope in the context of the MHC class I molecule H-2Kb. To make EG.7-OVA cells apoptosis resistant, they are tranduced with IAPs using the same lentiviral approach employed for K562 cells described above. These cells are injected into the mammary fat pad of wild-type B6 mice (which are syngeneic) to establish apoptosis-resistant tumors. CD8+ selected OT-I splenocytes are transduced with a three- component lentiviral construct identical to that employed above, save that the human genes will be replaced by their murine orthologs. The splenocytes are then adoptively transferred to mice, after which the mice are evaluated for tumor regression and overall survival after single or repeated doses of varying numbers of adoptively transferred lymphocytes. As controls, results are compared to those obtained using GzB-NULL modified OT-1 lymphocytes, and to results obtained in mice with tumors derived from IAP-negative (lymphocyte sensitive) and/or O VA-negative cells.

While the present invention has been described with reference to several particular example embodiments, those skilled in the art will recognize that many changes may be made thereto without departing from the spirit and scope of the present invention. The present invention is applicable to a variety of sensor implementations and other subject matter, in addition to those discussed above.

### Definitions

Unless otherwise specifically defined herein, terms and symbols of nucleic acid chemistry, biochemistry, genetics, and molecular biology used herein follow those of standard treatises and texts in the field, e.g. Kornberg and Baker, DNA Replication, Second Edition (W. H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Abbas et al, Cellular and Molecular Immuology, 6^{th} edition (Saunders, 2007); Murphy, Janeway's Immunobiology, 8^{th} edition (Garland Science).

“Antigen presenting cell” or “APC” means a cell that displays peptide fragments of protein antigens, in association with MHC molecules, on its surface, and activates antigen-specific T cells. For example, cytotoxic T cells are activated to generate an effector response.

“Apoptosis” means a process of cell death characterized by DNA cleavage, nuclear condensation and fragmentation, and plasma membrane blebbing that leads to phagocytosis of the cell without inducing an inflammatory response.

“Cytotoxic T-cell” or “cytotoxic T-lymphocyte” (“CTL”) means a type of T cell that secrets effector agents on recognition of a target cell by the specific binding its T cell receptor to an MHC molecule on the target cell complexed with an epitope. CTLs are characterized by the presence of the CD8 cell surface receptor.

“Cytotoxic lymphocyte” in one aspect means a lymphocyte capable of producing an effector response which, in some embodiments, comprises the formation and exocytosis of intracellular granules (i.e. “lytic granules”) containing effector agents; that is, a cytotoxic lymphocyte is lymphocyte having granzyme-perforin pathway functionality. In some embodiments, cytotoxic lymphocytes are CTLs or NK cells. In other aspects, the term “cytotoxic lymphocyte” may encompass any cell type that possesses (i) a molecular recognition system specific for a desired, or target, cell population, and (ii) a granzyme-perforin pathway that may be activated upon recognition of cells of the target cell population. As discussed above, molecular recognition systems include, TCRs, CARs, and the like, e.g. James et al, Nature, 487: 64-69 (2012).

“Epitope” or “target antigen of a cytotoxic lymphocyte” means any molecule that may be recognized in a specific manner by an antigen receptor, e.g. TCR, CAR, or the like, on a cytotoxic lymphocyte. In some embodiments, an epitope is a portion of a protein, i.e. a peptide, which binds to, or is complexed with, an MHC molecule for recognition by a cytotoxic lymphocyte. Such peptide epitopes may include posttranslational modifications, such as carbohydrate or lipid moieties. In some embodiments, epitopes may be peptides, polysaccharides, or lipids, or combinations thereof.

“Effector agent” means a constituent of a lytic granule. Lytic granules are described in Clark et al, Curr. Opin. Immunol., 15(3): 516-521 (2003); Page et al, Biochim Biophys. Acta, 1401(2): 146-156 (1998), which are incorporated herein by reference. In some embodiments, an effector agent is a compound released by a cytotoxic lymphocyte by exocytosis of a granule. In some embodiments, such exocytosis results from the specific binding of cytotoxic lymphocyte receptors to MHC-epitope complexes on the surface of a target cell. In other embodiments, such exocytosis results from the specific binding of chimeric antigen receptors (CARs) of a cytotoxic lymphocyte to CAR-specific molecules of a target cell, for example, cognate surface antigens of a target cell. In some embodiments, an effector agent, either alone or with other molecules, is capable of directly or indirectly triggering an apoptotic pathway of the target cell. In some embodiments, the term “effector agent” refers to protein effector agents, sometimes referred to as “lytic granule proteins,” such as a granzyme. Effector agents include, but are not limited to, granzymes, perforins, and granulysins. In other embodiments the effector agent may be an engineered protein, derived from a lytic granule component. That is, a fusion protein may comprise only a portion of an effector agent, such as, a minimal portion required for trafficking to a target cell. The latter embodiment would allow delivery of larger predetermined proteins to target cells.

“Effector response” or “effector function” in reference to a cytotoxic lymphocytes means activation of a granzyme-perforin pathway after recognition of an MHC-epitope or other surface molecule on a target cell. In some embodiments, activation of a granzyme-perforin pathway includes transport of lytic granules to the cell surface and exocytosis of effector agents of the lytic granules in the vicinity of the target cell, e.g. as described by Cullen et al, Cell Death and Differentiation, 15: 251-262 (2008); Trapani et al, Nature Reviews Immunology, 2: 735-747 (2002).

“Granzyme” or “granzyme protein” means a serine protease found in the granules of cytotoxic lymphocytes, such as CTLs and natural killer (NK) cells, that is released by exocytosis, enters target cells, mainly through perforin-created membrane pores, and proteolytically cleaves and activates caspases, which in turn cleave several substrates and induce target cell apoptosis. In some embodiments, a granzyme is a human granzyme. In other embodiments, a human granzyme includes granzymes A, B, H, K or M, separately or together. In other embodiments, a granzyme is a human granzyme B. Granzymes and their functions are described in Cullen et al, Cell Death and Differentiation, 15: 251-262 (2008); Symthe et al, J. Leukocyte Biol., 70: 18-29 (2001); and like references.

“Kit” means any delivery system for delivering materials or reagents for carrying out a method of the invention. In the context of reaction assays, such delivery systems include systems that allow for the storage, transport, or delivery of reaction reagents (e.g., probes, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. Such contents may be delivered to the intended recipient together or separately. For example, a first container may contain an enzyme for use in an assay, while a second container contains probes.

“Major histocompatibility complex” (“MHC”) molecule means a heterodimeric membrane protein encoded in the MHC locus that serves as a peptide display molecule for recognition by T lymphocytes. For MHC molecules of different samples of cells or tissue to be “matched” means that cells of the different samples express the same genetic variant of an MHC molecule.

“Memory T cells” mean T lymphocytes that mediate rapid and enhanced, i.e. “memory,” responses to second and subsequent exposure to antigens. Memory T lymphocytes are produced by antigen stimulation of naïve lymphocytes and survive in a functionally quiescent state for many years after the antigen is eliminated.

“Perforin” means a cellular membrane pore forming protein produced by cytotoxic lymphocytes upon stimulation of a cell death pathway, e.g. Trapani et al, Nature Reviews Immunology, 2: 735-747 (2002); Keefe et al, Immunity, 23: 249-262 (2005); Catalfamo et al, Curr. Opin. Immunol., 15: 522-527 (2003).

“Peptide” or “polypeptide” refers to a class of compounds composed of amino acid residues chemically bonded together by amide linkages with elimination of water between the carboxy group of one amino acid and the amino group of another amino acid. In some embodiments, peptides comprise a linear chain of amino acids having a length in the range of from 6 to 20 amino acids. In other embodiments, peptides comprise a linear chain of amino acids having a length in the range of from 8 to 14 amino acids.

“Polymerase chain reaction” or “PCR” means means a reaction for the in vitro amplification of specific DNA sequences by the simultaneous primer extension of complementary strands of DNA. In other words, PCR is a reaction for making multiple copies or replicates of a target nucleic acid flanked by primer binding sites, such reaction comprising one or more repetitions of the following steps: (i) denaturing the target nucleic acid, (ii) annealing primers to the primer binding sites, and (iii) extending the primers by a nucleic acid polymerase in the presence of nucleoside triphosphates. Usually, the reaction is cycled through different temperatures optimized for each step in a thermal cycler instrument. Particular temperatures, durations at each step, and rates of change between steps depend on many factors well-known to those of ordinary skill in the art, e.g. exemplified by the references: McPherson et al, editors, PCR: A Practical Approach and PCR2: A Practical Approach (IRL Press, Oxford, 1991 and 1995, respectively). For example, in a conventional PCR using Taq DNA polymerase, a double stranded target nucleic acid may be denatured at a temperature >90° C., primers annealed at a temperature in the range 50-75° C., and primers extended at a temperature in the range 72-78° C.

Reaction volumes typically range from a few hundred nanoliters, e.g. 200 nL, to a few hundred μL, e.g. 200 μL. “Primer” means an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process are determined by the sequence of the template polynucleotide. Usually primers are extended by a DNA polymerase. Primers usually have a length in the range of from 14 to 36 nucleotides.

“Transgene” means natural or artificially constructed genetic material that has been transferred naturally, or by any of a number of genetic engineering techniques, to an organism or from one organism to another.

“Transfection” and/or “transformation” and/or “transduction” are used synonymously herein mean the transfer of exogenous genetic material to a target mammalian cell. Such transfer may be result in temporary or transient expression of a transgene or temporary or transient transcription of an RNA, for example, because of exhaustion of genetic material, loss or degradation of genetic material, lack of replication of genetic material, or the like. In some embodiments, “transfection” means “stable transfection” as the latter term is commonly used, e.g. Kim et al, Anal. Bioanal. Chem., 379: 3173-3178 (2010). Exogenous genetic material may include plasmids, viral vectors, transgenes, transposons, or the like. “Stable” as used herein means that the exogenous genetic material persists through multiple cell divisions or for the life of the cellular host. The exogenous genetic material may be integrated into the genome of a target mammalian cell or it may comprise episomal DNA, such as a plasmid. Typically, transferred genetic material expresses one or more proteins of interest in a target mammalian cell.

“Vector” means a nucleic acid molecule capable of transporting another nucleic acid to which it has been linked. One type of vector is a “plasmid,” which refers to a circular double stranded DNA loop into which additional DNA segments may be ligated. Another type of vector is a phage vector. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “recombinant vectors”). In general, expression vectors of utility in recombinant DNA techniques are often in the form of plasmids. In the present specification, “plasmid” and “vector” may be used interchangeably as the plasmid is the most commonly used form of vector.

1. A method of delivering a compound to target cells, the method comprising the steps of
providing cytotoxic lymphocytes specific for the target cells and having a granzyme-perforin pathway;
genetically modifying the cytotoxic lymphocytes to express a fusion protein comprising an effector agent and a predetermined protein to form a population of delivery lymphocytes, such that the fusion protein is sequestered in lytic granules of the delivery lymphocyte;
contacting the target cells with the delivery lymphocytes so that granzyme-perforin pathways thereof are activated, thereby delivering the fusion protein to cytosols of the target cells.

2. The method of claim 1 wherein said step of contacting includes administering said delivery lymphocytes to an individual so that said delivery lymphocytes contact said target cells of the individual.

3. The method of claim 2 further including a step of expanding said population of delivery lymphocytes prior to said step of administration.

4. The method of claim 2 wherein said cytotoxic lymphocytes are matched with said target cells.

5. The method of claim 4 wherein said cytotoxic lymphocytes are autologous to said individual.

6. The method of claim 1 wherein said cytotoxic lymphocytes are cytotoxic T cells and wherein said contact of said delivery lymphocytes with said target cells includes binding of T cell receptors of said delivery lymphocytes with WHC molecules of said target cells.

7. The method of claim 1 wherein said step of genetically modifying includes stably transfecting said cytotoxic lymphocytes so that said cytotoxic lymphocytes express said fusion protein.

8. The method of claim 7 wherein said step of stably transfecting said cytotoxic lymphocytes is carried out with a viral vector.

9. The method of claim 8 wherein said viral vector is a lentivirus vector, an adenovirus vector or an adeno-associated viral vector.

10. The method of claim 1 wherein said step of genetically modifying includes inserting into genomes of said cytotoxic lymphocytes at least one transgene comprising said fusion protein.

11. The method of claim 10 wherein said effector agent is a granzyme or a portion thereof, a gramulysin or a portion thereof, a serglycin or a portion thereof, or a perforin or a portion thereof.

12. The method of claim 11 wherein said effector agent is grazyme A or a portion thereof or granzyme B or a portion thereof.

13. The method of claim 10 wherein said step of inserting includes generating a double stranded cleavage with one, or more programmable nucleases followed by homology-directed repair with a donor template comprising said at least one transgene.

14. The method of claim 13 wherein said programmable nuclease is an RNA-guided nuclease.

15. The method of claim 1 wherein said cytotoxic lymphocytes are primary NK cells or immortalized NK cells.

16. The method of claim 1 wherein said fusion protein comprises a granzyme and a therapeutic protein.

17. The method of claim 16 wherein said therapeutic protein is a toxin.

18. The method of claim 17 wherein said step of genetically modifying further includes genetically modifying said cytotoxic lymphocyte to express a protein capable of neutralizing effects of said toxin in said cytotoxic lymphocyte.

19. The method of claim 17 wherein said toxin is a Pseudomonas aeruginosa exotoxin A (PE), a diphtheria toxin (DT), a ricin, a saporin, or a fragment thereof.

20. The method of claim 1 wherein said fusion protein comprises a protein capable of binding a nucleic acid or a small molecule for delivery thereof to said cytosol of said target cells.

21. The method of claim 1 wherein said cytotoxic lymphocyte is further genetically modified to attenuate or eliminate expression of endogenous cytotoxic effector mechanisms, allowing said modified cytotoxic lymphocyte to be used to deliver beneficial therapeutic molecules.

22. A composition for delivering a predetermined protein to target cells comprising cytotoxic lymphocytes specific for the target cells, the cytotoxic lymphocytes being genetically modified to express a fusion protein comprising an effector agent and a predetermined protein, wherein the fusion protein is sequestered in lytic granules of the cytotoxic lymphocyte and deliverable to cytosols the target cells by a granzyme-perforin pathway of the cytotoxic lymphocytes whenever the cytotoxic lymphocytes contact the target cells.

23. The composition of claim 22 wherein said cytotoxic lymphocytes are MHC-matched with said target cells.

24. The composition of claim 22 wherein said cytotoxic lymphocytes are genetically modified by insertion of a transgene into the genomes thereof such that the transgene comprises said fusion protein.

25. The method of claim 22 wherein said effector agent is a granzyme or a portion thereof, a granulysin or a portion thereof, a serglycin or a portion thereof, or a perforin or a portion thereof.

26. The composition of claim 25 wherein said effector agent is a granzyme or a portion thereof.

27. The composition of claim 26 wherein said predetermined protein is a toxin.

28. The composition of claim 27 wherein said cytotoxic lymphocyte is further genetically modified to express a protein capable of neutralizing effects of said toxin in said cytotoxic lymphocyte.
